(19) United States
(12) Patent Application Publication
(10) Pub. No.: US 2023/0167505 A1
(43) Pub. Date: Jun. 1, 2023

(54) CIRCULATING TUMOR DNA AS A BIOMARKER FOR LEUKEMIA TREATMENT

(71) Applicant: Cardiff Oncology, Inc., San Diego, CA (US)

(72) Inventors: Errin SAMUELSZ, San Diego, CA (US); Mark ERLANDER, San Diego, CA (US); Maya RIDINGER, San Diego, CA (US)

(21) Appl. No.: 17/792,069

(22) PCT Filed: Jan. 13, 2021
(86) PCT No.: PCT/US21/13287

§ 371 Date: Jul. 11, 2022

Related U.S. Application Data
(60) Provisional application No. 62/960,166, filed on Jan. 13, 2020.

(51) Int. Cl. C12Q 1/6886

(52) U.S. Cl. CPC C12Q 1/6886; C12Q 2600/112; C12Q 2600/156

(57) ABSTRACT

Provided herein includes a method comprising analyzing circulating tumor DNA (ctDNA), for example ctDNA in plasma, from a patient with leukemia, to predict and/or determine clinical response. The leukemia can be, for example, acute myeloid leukemia (AML), myelodysplastic syndrome (MDS) or chronic melomonocytic leukemia (CMML).

### CROSS-REFERENCE TO RELATED APPLICATIONS

This application is a U.S. national phase application under 35 U.S.C. § 371 of International Application No. PCT/US2021/013287, filed on Jan. 13, 2021 and published as WO 2021/146322 A1 on Jul. 22, 2021, which claims the benefit of priority to U.S. Patent Application No. 62/960,166, filed on Jan. 13, 2020, the content of which is incorporated herein by reference in its entirety.

### BACKGROUND

### Field

The present application generally relates to methods for monitoring cancer treatment. More specifically, methods of using circulating tumor DNA to evaluate the status of a leukemia and the effectiveness of treatments for leukemia are provided.

### Description of the Related Art

The Polo-like kinase 1 (PLK1) is the most well characterized member of the 5 members of the family of serine/threonine protein kinases and strongly promotes the progression of cells through mitosis. PLK1 performs several important functions throughout mitotic (M) phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis (Stebhardt, 2010). It plays a key role in centrosome functions and the assembly of bipolar spindles. It also acts as a negative regulator of p53 family members leading to ubiquitination and subsequent degradation of p53/TP53, inhibition of the p73/TP73 mediated pro-apoptotic functions and phosphorylation/degradation of bora, a cofactor of Aurora kinase A. During the various stages of mitosis PLK1 localizes to the centrosomes, kinetochores and central spindle. PLK1 is a master regulator of mitosis and aberrantly overexpressed in a variety of human cancers including AML and is correlated with cellular proliferation and poor prognosis (Degenhardt, 2010). The first PLK1 inhibitor, BI 2536, showed interesting clinical activity in patients with relapsed and treatment refractory AML in an early clinical study, and its successor volasertib (also known as BI 6727) demonstrated a more favorable toxicity profile, as well as potent anti-leukemic activity as monotherapy and in combination with LDAC in heavily pretreated AML patients (Müller-Tidow et al., 2013; Schoffski et al., 2012; Döhner et al., 2014). In 2013, volasertib received a Breakthrough Therapy designation from the Food and Drug Administration (FDA) for its potential as a treatment for patients with untreated AML who are ineligible for intensive remission induction therapy.

Onvansertib (also known as PCM-075, NMS-1286937, NMS-937, “compound of formula (I)” in U.S. Pat. No. 8,927,530; IUPAC name 1-(2-hydroxyethyl)-8-{[5-(4-methylpiperazin-1-yl)-2-(trifluoromethoxy) phenyl]amino}-4,5-dihydro-1H-pyrazolo [4,3-h] quinazoline-3-carboxamide) is the first PLK1 specific ATP competitive inhibitor administered by oral route to enter clinical trials with proven antitumor activity in different preclinical models (Beria et al., 2011; Hartsink-Segers et al., 2013; Sero et al., 2014; Valsasina et al., 2012; Casolara et al., 2013).

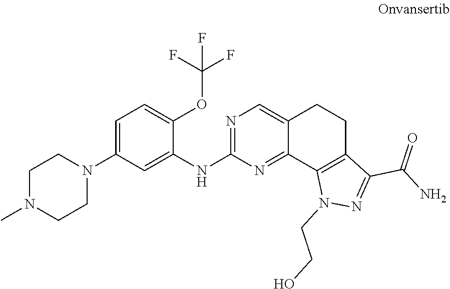 [SMILES: CN1CCN(c2ccc(OC(F)(F)F)c(Nc3ncc4c(n3)-c3c(c(C(N)=O)nn3CCO)CC4)c2)CC1]

Onvansertib shows high potency in proliferation assays having low nanomolar activity on a large number of cell lines, both from solid as well as hematologic tumors. Onvansertib potently causes a mitotic cell-cycle arrest followed by apoptosis in cancer cell lines and inhibits xenograft tumor growth with a clear PLK1-related mechanism of action at well tolerated doses in mice after oral administration. In addition, onvansertib shows activity in combination therapy with approved cytotoxic drugs, such as irinotecan, in which there is enhanced tumor regression in HT29 human colon adenocarcinoma xenografts compared to each agent alone (Valsasina et al., 2012; see also U.S. Pat. No. 8,927,530), and shows prolonged survival of animals in a disseminated model of AML in combination therapy with cytarabine (Valsasina et al., 2012; Casolaro et al., 2013). Onvansertib has favorable pharmacologic parameters and good oral bioavailability in rodent and nonrodent species, as well as proven antitumor activity in different nonclinical models using a variety of dosing regimens, which may potentially provide a high degree of flexibility in dosing schedules, warranting investigation in clinical settings. Onvansertib has several advantages over volasertib, including a higher degree of potency and specificity for the PLK1 isozyme, and oral bioavailability.

In acute myeloid leukemia (AML), blast cells carrying a driver mutation in the bone marrow (BM) migrate into circulation in the peripheral blood (PB). Hematologic mutations are detectable in a majority of AML patients, and are reported to persist in over 50% of patients during complete remission (CR) (Jongen-Lavrencic et al., 2018). Patients with relapsed or refractory AML (R/R AML) have limited treatment options and dismal outcome. There is a need to find effective treatment for leukemia and effective methods to evaluate the status of a leukemia and the effectiveness of treatments for leukemia.

### SUMMARY

Provided herein includes a method comprising analyzing ctDNA, for example ctDNA in plasma, from a patient with leukemia, including but not limited to acute myeloid leukemia (AML), myelodysplastic syndrome (MDS), and chronic melomonocytic leukemia (CMML).

Disclosed herein includes methods of determining responsiveness of a subject to a leukemia treatment. The method can comprise, for example, analyzing circulating tumor DNA (ctDNA) of a subject with leukemia, the subject is undergoing a treatment and/or has received a treatment for leukemia, thereby determining the responsiveness of the subject to the leukemia treatment. In some embodiments, determining the responsiveness of the subject comprises determining if the subject is a responder of the treatment, if the subject is or is going to be in CR, if the subject is or is going to be in incomplete hematologic recovery (CRi), if the subject is or is going to be in morphologic leukemia-free state (MLFS), or if the subject is or is going to be in partial remission (PR). For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the leukemia treatment.

In some embodiments, the first time point is prior or immediately prior to the leukemia treatment, and at least one of the one or more additional time points are at the end of or after at least a cycle of the leukemia treatment. In some embodiments, the cycle of the leukemia treatment is the first cycle of the leukemia treatment.

In some embodiments, the first time point is prior or immediately prior to a first cycle of the leukemia treatment, and the one or more additional time points are at the end of or after a second cycle of the leukemia treatment. In some embodiments, the first cycle of the leukemia treatment is immediately prior to the second cycle of the leukemia treatment. In some embodiments, the method comprises continuing the leukemia treatment to the subject if the subject is indicated as responsive to the leukemia treatment. In some embodiments, the method comprises discontinuing the leukemia treatment to the subject and/or starting a different leukemia treatment to the subject if the subject is not indicated as responsive to the leukemia treatment.

Disclosed herein include methods of determining leukemia status of a subject, comprising analyzing circulating tumor DNA (ctDNA) of a subject, thereby determining leukemia status of the subject. The subject can be a subject undergoing a current treatment for leukemia, a subject that has received a prior treatment for leukemia, and/or a subject that is in remission for leukemia. The subject in remission for leukemia can be in complete remission (CR), in CR with incomplete hematologic recovery (CRi), in morphologic leukemia-free state (MLFS), or in partial remission (PR).

In some embodiments, analyzing the ctDNA comprises detecting variant allele frequency in the ctDNA. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, an increase in the variant allele frequency at the additional sample(s) relative to the first sample indicates that the subject is at risk of leukemia relapse or is in leukemia relapse.

In some embodiments, the first time point is prior or immediately prior to the leukemia treatment, and the one or more additional time points are at the end of or after at least a cycle of the leukemia treatment, optionally the cycle of the leukemia treatment is the first cycle of the leukemia treatment.

In some embodiments, the first time point is prior or immediately prior to a first cycle of the leukemia treatment, and the one or more additional time points are at the end of or after a second cycle of the leukemia treatment, optionally the first cycle of the leukemia treatment is immediately prior to the second cycle of the leukemia treatment.

In some embodiments, the method comprises starting an additional leukemia treatment to the subject if the subject is indicated as in leukemia relapse. The additional leukemia treatment can be the same or different from the current or prior leukemia treatment.

The variant allele frequency in ctDNA can be determined, for example, by total mutation count in the ctDNA in each of the first sample and one or more additional samples, or by the mean variant allele frequency in each of the first sample and one or more additional samples. In some embodiments, the variant allele frequency is mutant allelic frequency (MAF) for a driver mutation of leukemia. In some embodiments, the variant allele frequency is MAF for one or more driver mutations of leukemia. In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is, or is about, 0.01 to −0.10. In some embodiments, the MAF threshold is, or is about, 0.06. In some embodiments, the MAF threshold is, or is about, 0.05.

In some embodiments, the first sample comprises ctDNA from the subject before treatment, and the one of additional samples comprises ctDNA from the subject after treatment. In some embodiments, the driver mutation is a mutation in one of the 75 genes set forth in Table 3. In some embodiments, at least one of the one or more the driver mutations is a mutation in in the 75 genes set forth in Table 3. In some embodiments, one or more the driver mutations are mutations in the 75 genes set forth in Table 3.

The driver mutation or at least one of the one or more driver mutations can be in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the driver mutation or at least one of the one or more driver mutations is one or more of the mutations set forth in Table 12 as HGVSc. In some embodiments, the method further comprises determining variant allele frequency in one or more of the ctDNA, PBMCs and BMMCs of the subject.

The ctDNA can be analyzed using, for example, polymerase chain reaction (PCR), next generation sequencing (NGS), and/or droplet digital PCR (ddPCR).

The type of the leukemia can vary. For example, the leukemia can be advanced, metastatic, refractory, and/or relapsed. In some embodiments, the leukemia is acute myeloid leukemia (AML), myelodysplastic syndrome (MDS) or chronic melomonocytic leukemia (CMML). In some embodiments, the leukemia is relapsed or refractory acute myeloid leukemia.

The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the leukemia treatment comprises standard of care therapies for leukemia. In some embodiments, the leukemia treatment comprises administration (for example, oral administration) of a Polo-like kinase 1 (PLK1) inhibitor (for example, onvansertib). In some embodiments, the treatment comprises administration of onvansertib for five days in a cycle of 21 to 28 days. In some embodiments, the treatment comprises administration of onvansertib at 12 mg/m²-90 mg/m². In some embodiments, a maximum concentration (Cₘₐₓ) of onvansertib in a blood of the subject is from about 100 nmol/L to about 1500 nmol/L. In some embodiments, an area under curve (AUC) of a plot of a concentration of onvanserib in a blood of the subject over time is from about 1000 nmol/L·hour to about 400000 nmol/L·hour. In some embodiments, a time (Tₘₐₓ) to reach a maximum concentration of onvansertib in a blood of the subject is from about 1 hour to about 5 hours. In some embodiments, an elimination half-life (T_{1/2}) of onvansertib in a blood of the subject is from about 10 hours to about 60 hours. In some embodiments, the leukemia treatment comprises at least one additional administration of cancer therapeutics or cancer therapy. The PLK inhibitor and the cancer therapeutics or cancer therapy can, for example, co-administered simultaneously or sequentially. In some embodiments, the additional cancer therapeutics is cytarabine, low-dose cytarabine (LDAC) and/or decitabine. In some embodiments, the treatment comprises administration of LDAC at 20 mg/m² subcutaneous (SC) once a day (qd) for ten days in a cycle, and/or the treatment comprises administration of decitabine at 20 mg/m² intravenous (IV) qd for five days in a cycle.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include methods of improving treatment outcome for leukemia. The method can comprise: detecting variant allele frequency in circulating tumor DNA (ctDNA) obtained from a subject at a first time point in a first sample before the subject undergoes a leukemia treatment; detecting variant allele frequency in ctDNA obtained from the subject at one or more additional time points in one or more additional samples after the subject undergoes the leukemia treatment; determining the difference of the variant allele frequency in ctDNA between the first and at least one of the one or more additional samples, a decrease in the variant allele frequency in at least one of the additional samples relative to the first sample indicates the subject as responsive to the leukemia treatment; and continuing the leukemia treatment to the subject if the subject is indicated as responsive to the leukemia treatment, or discontinuing the leukemia treatment to the subject and/or starting a different leukemia treatment to the subject if the subject is not indicated as responsive to the leukemia treatment.

Also disclosed herein include methods of treating leukemia. The method can comprise: administering a leukemia treatment to a subject in need thereof; determining a decrease, relative to a variant allele frequency in a first sample of the subject obtained at a first time point before the subject receives the leukemia treatment, in a variant allele frequency in a second sample of the subject obtained at a second time point after the subject receives the leukemia treatment; and continuing with the leukemia treatment. In some embodiments, the subject is a subject newly diagnosed with leukemia, for example a subject that has not received any prior cancer treatment before the leukemia treatment. In some embodiments, the subject has received prior cancer treatment and was in remission for leukemia, for example a subject in complete remission (CR), in CR with incomplete hematologic recovery (CRi), in morphologic leukemia-free state (MLFS), or in partial remission (PR) after receiving the prior cancer treatment.

The first time point can be, for example, prior or immediately prior to the leukemia treatment. The at least one of the one or more additional time points can be, for example, at the end of or after at least a cycle of the leukemia treatment. In some embodiments, the cycle of the leukemia treatment is the first cycle of the leukemia treatment. In some embodiments, the first time point is prior or immediately prior to a first cycle of the leukemia treatment, and the one or more additional time points are at the end of or after a second cycle of the leukemia treatment. In some embodiments, the first cycle of the leukemia treatment is immediately prior to the second cycle of the leukemia treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of leukemia. In some embodiments, the variant allele frequency is mutant allelic frequency (MAF) for one or more driver mutations of leukemia. In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is −0.05.

The driver mutation can be, for example, a mutation in one of the 75 genes set forth in Table 3, at least one of the one or more the driver mutations is a mutation in in the 75 genes set forth in Table 3, and/or one or more the driver mutations are mutations in the 75 genes set forth in Table 3. In some embodiments, the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the driver mutation or at least one of the one or more driver mutations is one or more of the mutations set forth in Table 12 as HGVSc.

In some embodiments, the method further comprises determining variant allele frequency in one or more of the ctDNA, PBMCs and BMMCs of the subject. The variant allele frequency in ctDNA can be detected, for example, using polymerase chain reaction (PCR) or next generation sequencing (NGS). In some embodiments, the variant allele frequency in ctDNA is detected using droplet digital PCR (ddPCR).

The leukemia can be advanced, metastatic, refractory, and/or relapsed. In some embodiments, the leukemia is acute myeloid leukemia (AML), myelodysplastic syndrome (MDS) or chronic melomonocytic leukemia (CMML). In some embodiments, the leukemia is relapsed or refractory acute myeloid leukemia.

At least one of the first sample, the one or more additional samples, and the second sample can be derived from whole blood of the subject, plasma of the subject, serum of the subject, or a combination thereof. In some embodiments, the ctDNA is from whole blood of the subject, plasma of the subject, serum of the subject, or a combination thereof. In some embodiments, the leukemia treatment comprises standard of care therapies for leukemia.

In some embodiments, the leukemia treatment comprises administration (e.g., oral administration) of a Polo-like kinase 1 (PLK1) inhibitor (e.g., onvansertib). The treatment can comprise administration of onvansertib for five days in a cycle of 21 to 28 days. In some embodiments, the treatment comprises administration of onvansertib at 12 mg/m²-90 mg/m². In some embodiments, a maximum concentration (Cₘₐₓ) of onvansertib in a blood of the subject is from about 100 nmol/L to about 1500 nmol/L. In some embodiments, an area under curve (AUC) of a plot of a concentration of onvanserib in a blood of the subject over time is from about 1000 nmol/L·hour to about 400000 nmol/L·hour. In some embodiments, a time (Tₘₐₓ) to reach a maximum concentration of onvansertib in a blood of the subject is from about 1 hour to about 5 hours. In some embodiments, an elimination half-life (T_{1/2}) of onvansertib in a blood of the subject is from about 10 hours to about 60 hours.

In some embodiments, the leukemia treatment comprises at least one additional administration of cancer therapeutics or cancer therapy. For example, the PLK inhibitor and the cancer therapeutics or cancer therapy can be co-administered simultaneously or sequentially. In some embodiments, the additional cancer therapeutics is cytarabine, low-dose cytarabine (LDAC) and/or decitabine. In some embodiments, the leukemia treatment comprises administration of LDAC at 20 mg/m² subcutaneous (SC) once a day (qd) for ten days in a cycle, and/or the treatment comprises administration of decitabine at 20 mg/m² intravenous (IV) qd for five days in a cycle.

The leukemia treatment can comprise one or more cycles. In some embodiments, variant allele frequency in ctDNA is detected before, during and after each cycle of the leukemia treatment. In some embodiments, each cycle of treatment is at least 21 days. In some embodiments, each cycle of treatment is from about 21 days to about 28 days. In some embodiments, the subject is human.

### BRIEF DESCRIPTION OF THE DRAWINGS

FIG. 1. Dosing design for phase 1b/2 study of onvansertib described in Example 1.

FIGS. 2A-D. Clinical course and bone marrow response of patients evaluable for efficacy (N=36). FIGS. 2A and 2B, Response to treatment, duration of treatment, and dose adjustments are shown on swimmer plots at the different dose levels (onvansertib 12-90 mg/m²) for both arms. FIGS. 1C and 1D, Waterfall plots of percent change in bone marrow blast from baseline (best response) in patients from both arms. Patient identifiers and onvansertib doses (12-90 mg/m²) are indicated.

FIGS. 3A-B. Mutant allele frequency (MAF) monitoring in ctDNA isolated from plasma. FIG. 3A, ctDNA analysis workflow. FIG. 3B, Correlation between MAF measured by digital droplet PCR (ddPCR) in ctDNA isolated from plasma and mononuclear cells from bone marrow biopsies at matched timepoints (n=49, r2=0.91, p<0.0001). PBMCs=peripheral blood mononuclear cells; BMMCs=bone marrow mononuclear cells; NGS=next generation sequencing.

FIGS. 4A-C. ctDNA MAF monitoring as a predictive biomarker of clinical response. MAF was measured by ddPCR in plasma ctDNA of 20 patients. FIG. 4A, Changes in MAF after the first cycle were calculated as log₂(C₁/C₀). Target used for each patient is indicated. FIG. 4B, ROC curve for clinical response prediction, showing the sensitivity and specificity of plasma ctDNA decrease after one cycle of treatment to predict clinical response (CR/CRi). FIG. 4C, Patients with decrease in ctDNA MAF at first cycle, using the optimal threshold of the ROC curve [log₂(C₁/C₀)=−0.06], were considered responders on the basis of ctDNA plasma test. Sensitivity, specificity, positive predictive value (PPV), and negative predictive value (NPV) of the ctDNA plasma test to predict clinical response are reported.

FIG. 5A-C. Plots showing high correlation of plasma-derived ctDNA mutation allelic frequencies (MAF) with MAF from bone marrow mononuclear cells.

FIG. 6. A log plot showing range of ctDNA MAF by response.

FIG. 7A-F. Individual patient evaluation for treatment response using % BM blast cells and ddPCR ctDNA % MAF.

FIGS. 8A-D. Onvansertib inhibitory activity in plasma and circulating blasts. TCTP and pTCTP protein levels were analyzed by Western blot analysis and pTCTP level was normalized to total TCTP level. FIGS. 8A-B, Plasma inhibitory activity assay was performed by incubating HL-60 cells with patient plasma samples. A, Immunoblot and its quantification demonstrate changes in pTCTP level with onvansertib-containing plasma samples. B, Graph represents the % pTCTP inhibition at the different onvansertib dose levels (n=3 or 4 patients per dose level). FIG. 8C, Immunoblot of pTCTP and TCTP using PBMCs isolated from blood samples collected on day 1 at predose (0 h) and 3 hours postdose (3 h) from target engagement patients (≥50% pTCTP inhibition, top) and nontarget engagement patients (bottom). Onvansertib dose level (12-40 mg/m²) and patient identifiers (in green for LDAC arm and purple for decitabine arm) are indicated. FIG. 8D, Waterfall plot of percent change in bone marrow blast from baseline (best response) in target engagement and nontarget engagement patients. Best clinical response (CR, CRi, and MLFS) and percent peripheral blasts (PB) at baseline are indicated. D, day; X h, number of hours posttreatment.

FIG. 9. Clinical course in phase 2 patients completing ≥1 cycle.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients (e.g., cyclosporine analogues, including CRV431).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

Disclosed herein include methods, compositions, kits, and systems for monitoring cancer treatment, for determining responsiveness of a subject to a cancer treatment, for determining cancer status in a subject, for improving treatment outcome for cancer, and for treatment cancer (e.g., leukemia). The methods, compositions, kits and systems disclosed herein can be used to guide cancer treatment, provide treatment recommendations, reduce or avoid unnecessary ineffective treatment for patients, and to reduce cancer treatment costs. For example, status, clinical responsiveness and/or prognosis of a treatment for leukemia and related diseases in a patient can be determined by evaluating changes in the patient's ctDNA. Provided herein includes a method comprising analyzing ctDNA, for example ctDNA in plasma, from a patient with leukemia and related diseases. These methods are expected to be useful for evaluating any leukemia, including but are not limited to, acute lymphocytic leukemia (ALL), chronic lymphocytic leukemia (CLL), chronic myelogenous leukemia (CML), AML, MDS, and CMML. In some embodiments, the leukemia is AML, MDS or CMML. The methods, composition, kits, and systems disclosed herein can be used as a non-invasive alternative to the current bone marrow biopsy-based methods, composition, kits, and systems for diagnostics, treatment recommendation, monitoring treatment effectiveness, providing guidance for adjustment in treatment, etc., for cancer (e.g., leukemia) patients. In some embodiments, the methods, composition, kits, and systems disclosed herein can be used in combination with the current bone marrow biopsy-based methods, composition, kits, and systems

### Leukemia and PLK Inhibitors

Acute myeloid leukemia (AML) is characterized by the clonal expansion of myeloid blasts resulting in bone marrow failure. AML is predominantly a disease of older patients with a median age at diagnosis of 68 years. For patients with AML who are believed unfit for, or do not desire, intensive treatment, hypomethylating agents (HMA; e.g., azacitidine or decitabine) or low-dose cytarabine (LDAC) have historically been treatment options. However, complete responses are uncommon and often of limited duration. In 2018, new agents (venetoclax and glasdegib) were approved in the U.S. in the first-line setting in combination with HMAs or LDAC for older and unfit patients based on phase II open-label trials. Recent updates from the ongoing randomized phase III studies showed significant increase in overall survival (OS) for venetoclax in combination with azacitidine, but not LDAC. Patients with relapsed or refractory (R/R) AML have very limited effective therapy options, particularly in the absence of targetable mutations such as FLT3 or IDH1/2, and their outcomes are dismal with median survival of less than 6 months.

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. PLK1 has been shown to be overexpressed in solid tumors and hematologic malignancies, including AML. PLK1 inhibition induces G2-M-phase arrest with subsequent apoptosis in cancer cells, and has emerged as a promising targeted therapy. Several PLK inhibitors have been studied in clinical trials. In a randomized phase II study of patients with AML who were treatment naïve yet unsuitable for induction therapy, the pan-PLK inhibitor, volasertib (BI6727), administered intravenously in combination with LDAC showed a significant increase in OS when compared with LDAC alone. A subsequent randomized phase III study identified no benefit of the combination and described an increased risk of severe infections.

Onvansertib (also known as PCM-075 or NMS-1286937) is a selective ATP-competitive PLK1 inhibitor. Biochemical assays demonstrated high specificity of onvansertib for PLK1 among a panel of 296 kinases, including other PLK members. Onvansertib has potent in vitro and in vivo antitumor activity in models of both solid and hematologic malignancies. Onvansertib inhibited cell proliferation at nanomolar concentrations in AML cell lines and tumor growth in xenograft models of AML. In addition, onvansertib significantly increased cytarabine antitumor activity in disseminated models of AML.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window.

As described herein, safety, pharmacokinetics, and preliminary clinical activity of onvansertib in combination with either LDAC or decitabine were determined in patients with R/R AML. Pharmacodynamics and biomarker studies, including baseline genomic profiling, serial monitoring of mutant allele fractions in plasma, and the extent of PLK1 inhibition in circulating blasts, were performed to identify biomarkers associated with clinical response.

Circulating Tumor DNA (ctDNA) and Detection Thereof

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. cfDNA can be obtained from a bodily fluid without the need to perform an in vitro cell lysis step, and thus presents a non-invasive option for genomic analysis. Provided herein include methods, compositions, kits and systems for detecting and/or analyzing cell free nucleic acids (e.g., ctDNA) in bodily fluid (e.g., peripheral blood) for clinical outcome prediction/determination. The methods can comprise combined analysis of single cells and cell-free nucleic acids. Provided herein include methods utilizing ctDNA from whole blood (e.g., plasma and/or serum) for therapeutic monitoring, and minimal/molecular residual disease determination.

Various assays (e.g., sequencing assays) can be used to detect and analyze ctDNA. The methods provided herein can comprise isolation and analysis of ctDNA from the blood (plasma and/or serum) of a subject of interest (e.g., a subject with leukemia, or a subject is in remission of leukemia, or a subject suspected to have leukemia), employing the use of molecular barcoding and sequencing as a readout. The method can comprise isolating plasma and ctDNA from intact cell-depleted blood. The method can comprise centrifugation to generate plasma and extraction of nucleic acids from plasma, followed by library prep with barcoding, sequencing, and then analysis. The ctDNA, for example, can be obtained from a plasma sample by known methods, and can be analyzed by methods including but not limited to polymerase chain reaction (PCR) and next generation sequencing (NGS). In some embodiments, the ctDNA is analyzed using droplet digital PCR (ddPCR).

The ctDNA can be from a bodily fluid (e.g., a blood sample) of a subject of interest (e.g., a subject with leukemia, or a subject is in remission of leukemia, or a subject suspected to have leukemia). The ctDNA can be obtained from plasma fraction, serum fraction, or both, of the blood sample. In some embodiments, the bodily sample is whole blood, serum, plasma, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine, or any combination thereof. In some embodiment, the ctDNA is obtained from blood and fractions thereof. A sample can be in the form originally isolated from a subject or can have been subjected to further processing to remove or add components, such as cells, or enrich for one component relative to another. Thus, a preferred body fluid for analysis is plasma or serum containing ctDNA. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may not have cancer or a detectable cancer symptom. The subject may have been treated with one or more cancer therapy, e.g., any one or more of chemotherapies, antibodies, vaccines or biologics. The subject may be in remission. The subject may be suspected to have cancer or any cancer-associated genetic mutations/disorders.

The ctDNA can carry one or more types of mutations, for example, germline mutations, somatic mutations, or both. Germline mutations refer to mutations existing in germline DNA of a subject. The ctDNA from a subject can carry one or more mutations in one or more genes, for example known cancer-associated genes (e.g., genes known to be associated leukemia). Somatic mutations refer to mutations originating in somatic cells of a subject, e.g., cancer cells. In some embodiments, the mutation can be cancer-associated mutations (e.g., cancer-associated somatic mutations).

Exemplary amounts of ctDNA in a biological sample (e.g., plasma or serum) before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of ctDNA molecules. The method can comprise obtaining 1 femtogram (fg) to 200 ng ctDNA. The ctDNA can have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

ctDNA can be isolated from bodily fluids (e.g., plasma) through a fractionation or partitioning step in which ctDNA, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. After such processing, samples can include various forms of nucleic acid including double stranded DNA and single stranded DNA. In some embodiments, single stranded DNA can be converted to double stranded forms so they are included in subsequent processing and analysis steps.

There are provided, in some embodiments, methods, reagents, compositions, and systems for analyzing complex genomic material while reducing or eliminating loss of molecular characteristic (e.g., epigenetic or other types of structural) information that is initially present in the complex genomic material. In some embodiments, molecular tags can be used to track ctDNA and determine genetic modifications (e.g., SNVs, indels, gene fusions and copy number variations). The method for detecting and analyzing ctDNA can comprise: classifying one or more variant properties derived from the sequence reads generated from one or more sequencing assays on the isolated cfNA as a true cancer-associated variant, a clonal hematopoiesis of indeterminate potential (CHIP)-associated variant, and/or a mutation of unknown origin. The method can comprise: adjusting the prediction score, the MRD score, and/or the efficacy score based on the classification of the one or more variant properties derived from the sequence reads generated from one or more sequencing assays on the isolated ctDNA.

Methods, compositions, kits and systems disclosed herein can be applied to different types of subjects. For example, the subject can be a subject receiving a cancer treatment (e.g., for leukemia), a subject at cancer remission (e.g., remission for leukemia), a subject has received one or more cancer treatment, or a subject suspected of having cancer (e.g., leukemia). The subject can have a stage I cancer, a stage II cancer, a stage III cancer, and/or a stage IV cancer. The cancer can comprise a hematological cancer, for example leukemia. Non-limiting examples of leukemia include acute lymphocytic leukemia (ALL), acute myeloid leukemia (AML), chronic lymphocytic (CLL), chronic myeloid (CML), chronic myelomonocytic (CMML), and a combination thereof. The methods can comprise: administering a therapeutic intervention to the subject. The therapeutic intervention can comprise a different therapeutic intervention, an antibody, an adoptive T cell therapy, a chimeric antigen receptor (CAR) T cell therapy, an antibody-drug conjugate, a cytokine therapy, a cancer vaccine, a checkpoint inhibitor, radiation therapy, surgery, a chemotherapeutic agent, or any combination thereof. The therapeutic intervention can be administered at a time when the subject has an early-stage cancer, and wherein the therapeutic intervention is more effective that if the therapeutic intervention were to be administered to the subject at a later time.

As disclosed herein, useful information such as the status of the leukemia can be obtained by evaluating ctDNA from more than one plasma sample, for example plasma collected (a) before or at treatment, and (b) at least once after treatment has started. In those embodiments, the second or subsequent samples can be taken at any time after treatment has started, e.g., after the first round of treatment, after multiple rounds of treatment, or after the leukemia is no longer detected in order to determine whether the leukemia has returned. See Example, where multiple plasma samples were evaluated in conjunction with bone marrow and peripheral blood cells. In some embodiments, the blood sample (e.g., plasma) collected after treatment is collected after a first round of treatment, e.g., at least 10, 15, 20, 21, 28, or 35 days, or any number of days in between or outside of those numbers, after the start of treatment.

In some embodiments, the ctDNA analysis measures the amount of a mutation in the ctDNA that is a driver mutation of the AML. In between embodiments, the ctDNA analysis measures mutation allelic frequencies (MAF) in the ctDNA.

As described herein, a decrease in MAF during treatment, e.g., when comparing the MAF before treatment with the MAF after the first round of treatment, is indicative or predictive of clinical response. In some embodiments, the decrease in ctDNA MAF can be considered as any amount of decrease, taking into consideration the type I vs. type II error to be tolerated. In some embodiments, a decrease in ctDNA MAF is measured as Log₂(C₁/C₀)<−0.05, where C0 is ctDNA MAF before treatment and C1 is ctDNA MAF after treatment. MAF determination can be used in the methods, compositions, kits and systems described herein for ctDNA analysis herein to determine effectiveness of treatment and future treatment of leukemia. In some embodiments, these methods can be used to decide whether to continue a treatment, e.g., when there is a decrease in MAF during the treatment, or change the treatment, e.g., if there is not a decrease in MAF during treatment.

### Methods for Predicting/Determining Treatment Efficacy and Status for Cancer

Disclosed herein include methods, compositions, kits, and systems for predicting/determining clinical outcome for cancer treatment, monitoring cancer treatment, predicting/determining responsiveness of a subject to a cancer treatment, determining cancer status in a subject, and improving cancer treatment outcome. Also disclosed herein include methods for treating cancer (e.g., leukemia). The methods, compositions, kits and systems can be used to guide cancer treatment, provide treatment recommendations, reduce or avoid unnecessary ineffective treatment for patients. ctDNA can be analyzed in the methods disclosed herein to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment.

In some embodiments, the subject is undergoing treatment for the leukemia. The treatment can be any treatment now known or later discovered for the leukemia. In various embodiments, the treatment is standard of care therapies for the leukemia. Nonlimiting examples of treatments that the methods disclosed herein can be used to include mutation-targeted treatments, e.g., onvansertib, and other treatments known to be effective, e.g., venetoclax, azacitidine, cytarabine (e.g., low-dose cytarabine [LDAC]), or decitabine. In some embodiments, the treatment comprises administration of onvansertib. In some embodiments, the administration of onvansertib is combined with low-dose cytarabine or decitabine.

Analyzing ctDNA can comprises analyzing ctDNA for one or more markers (e.g., ctDNAs comprising variant/mutant alleles). For example, ctDNA can be analyzed to assess variant allele frequency (VAF), change in the mean VAF, total mutation burden, and/or development of new driver mutations in a subject with cancer (e.g., leukemia). The subject can a subject to be selected for a cancer treatment, a subject that is undergoing a cancer treatment, or a subject that has undergone a cancer treatment. As used herein, a driver mutation refers to a mutation (e.g., a mutation within a gene) that confers a growth advantage to a cancer (e.g., leukemia). A driver mutation can drive the development of cancer. A driver mutation can be a somatic origin, or germline origin. In some embodiments, the driver mutation is a somatic mutation. In some embodiments, the driver mutation is a germline mutation. For the methods disclosed herein, analyzing ctDNA from a subject can comprise detecting variant allele frequency (VAF) in the ctDNA, and a change in VAF at different time points can indicate the subject as responsive to the leukemia treatment.

Some embodiments disclosed herein provide methods of predicting/determining responsiveness of a subject to a leukemia treatment. The method can comprise, for example, analyzing circulating tumor DNA (ctDNA) of a subject with leukemia, the subject is undergoing a treatment and/or has received a treatment for leukemia, thereby predicting/determining the responsiveness of the subject to the leukemia treatment. Predicting/determining the responsiveness of the subject can comprise, for example, predicting/determining if the subject is a responder of the treatment, predicting/determining if the subject is or is going to be in CR, predicting/determining if the subject is or is going to be in incomplete hematologic recovery (CRi), predicting/determining if the subject is or is going to be in morphologic leukemia-free state (MLFS), or predicting/determining if the subject is or is going to be in partial remission (PR). In some embodiments, analyzing ctDNA comprises detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the leukemia treatment.

The first time point can be, for example, prior or immediately prior, to the leukemia treatment. The at least one of the one or more additional time points can be, for example, at the end, or right before the end, of or after at least a cycle (e.g. the first cycle, the second cycle, the third cycle, or any of the subsequent cycles) of the leukemia treatment. In some embodiments, the cycle of the leukemia treatment is the first cycle of the leukemia treatment. In some embodiments, the first time point is prior or immediately prior to a first cycle of the leukemia treatment. In some embodiments, the one or more additional time points are at the end, or right before the end, of a second cycle, a third cycle, a fourth cycle, and/or a fifth cycle of the treatment. In some embodiments, the one or more additional time points are after a second cycle, a third cycle, a fourth cycle, and/or a fifth cycle of the leukemia treatment. In some embodiments, the first cycle of the leukemia treatment is immediately prior (e.g., one day, two days, three days, four days, or five days) to the second cycle of the leukemia treatment. In some embodiments, the method comprises continuing the leukemia treatment to the subject if the subject is indicated as responsive to the leukemia treatment. In some embodiments, the method comprises discontinuing the leukemia treatment to the subject and/or starting a different leukemia treatment to the subject if the subject is not indicated as responsive to the leukemia treatment.

Some embodiments disclosed herein include methods of determining leukemia status of a subject. The methods can comprise analyzing ctDNA of a subject, thereby determining leukemia status of the subject. The subject can be, for example, a subject undergoing a current treatment for leukemia, a subject that has received a prior treatment for leukemia, and/or a subject that is in remission for leukemia. The subject in remission for leukemia can be in complete remission (CR), in CR with incomplete hematologic recovery (CRi), in morphologic leukemia-free state (MLFS), or in partial remission (PR). In some embodiments, the ctDNA of the subject is analyzed for detecting variant allele frequency in the ctDNA. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, an increase in the variant allele frequency at at least one of the one or more additional samples relative to the first sample indicates that the subject is at risk of leukemia relapse or is in leukemia relapse. The ctDNA can be analyzed using, for example, polymerase chain reaction (PCR), next generation sequencing (NGS), and/or droplet digital PCR (ddPCR).

The first time point can be, for example, prior or immediately prior to the leukemia treatment. The one or more additional time points can be, for example, at the end of and/or after at least a cycle of the leukemia treatment, for example the first cycle of the leukemia treatment, the second cycle of the leukemia treatment, the third cycle of the leukemia treatment, the fourth cycle of the leukemia treatment, and/or the fifth cycle of the leukemia treatment. In some embodiments, the first time point is prior or immediately prior to a first cycle of the leukemia treatment. In some embodiments, the one or more additional time points are at the end of or after a second cycle of the leukemia treatment, where optionally the first cycle of the leukemia treatment is immediately prior to the second cycle of the leukemia treatment. In some embodiments, the one or more additional time points are at the end of and/or after a third cycle, a fourth cycle, and/or a fifth cycle of the leukemia treatment.

In some embodiments, the method comprises starting an additional leukemia treatment to the subject if the subject is indicated as in leukemia relapse. The additional leukemia treatment can be the same or different from the current or prior leukemia treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of leukemia. In some embodiments, the variant allele frequency is MAF for one or more driver mutations of leukemia. In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is −0.05.

The first sample can comprise ctDNA from the subject at various time points, for example before the treatment or during the treatment. In some embodiments, the first sample comprises ctDNA from the subject before the treatment (for example, immediately before the first cycle of the treatment). In some embodiments, the first sample comprises ctDNA from the subject before the second cycle of the treatment (for example, after the completion of the first cycle of the treatment and immediately before the second cycle of the treatment). The additional samples can comprise ctDNA from the subject during and/or after the treatment. In some embodiments, the additional samples comprise ctDNA from the subject right before the end of and/or after the treatment. In some embodiments, the additional samples comprise ctDNA from the subject right before the end of and/or after the first cycle, the second cycle, the third cycle, the fourth cycle, and/or the fifth cycle of the treatment.

Also disclosed herein include methods of improving treatment outcome for leukemia. The method can comprise: detecting variant allele frequency in circulating tumor DNA (ctDNA) obtained from a subject at a first time point in a first sample before the subject undergoes a leukemia treatment; detecting variant allele frequency in ctDNA obtained from the subject at one or more additional time points in one or more additional samples after the subject undergoes the leukemia treatment; determining the difference of the variant allele frequency in ctDNA between the first and at least one of the one or more additional samples, a decrease in the variant allele frequency in at least one of the additional samples relative to the first sample indicates the subject as responsive to the leukemia treatment; and continuing the leukemia treatment to the subject if the subject is indicated as responsive to the leukemia treatment, or discontinuing the leukemia treatment to the subject and/or starting a different leukemia treatment to the subject if the subject is not indicated as responsive to the leukemia treatment.

Methods of treating leukemia are also provided herein. The method can comprise, for example: administering a leukemia treatment to a subject in need thereof; determining a decrease, relative to a variant allele frequency in a first sample of the subject obtained at a first time point before the subject receives the leukemia treatment, in a variant allele frequency in a second sample of the subject obtained at a second time point after the subject receives the leukemia treatment; and continuing with the leukemia treatment. In some embodiments, the subject is a subject newly diagnosed with leukemia, for example a subject that has not received any prior cancer treatment before the leukemia treatment. In some embodiments, the subject has received prior cancer treatment and was in remission for leukemia, for example a subject in complete remission (CR), in CR with incomplete hematologic recovery (CRi), in morphologic leukemia-free state (MLFS), or in partial remission (PR) after receiving the prior cancer treatment.

The first time point can be, for example, prior or immediately prior to the leukemia treatment. The at least one of the one or more additional time points can be, for example, at the end of or after at least a cycle of the leukemia treatment. In some embodiments, the cycle of the leukemia treatment is the first cycle of the leukemia treatment. In some embodiments, the first time point is prior or immediately prior to a first cycle of the leukemia treatment, and the one or more additional time points are at the end of or after a second cycle of the leukemia treatment. In some embodiments, the first cycle of the leukemia treatment is immediately prior to the second cycle of the leukemia treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of leukemia. In some embodiments, the variant allele frequency is mutant allelic frequency (MAF) for one or more driver mutations of leukemia. In some embodiments, Log₂(C₁/C₀)<a MAF threshold indicates a decrease in ctDNA MAF C₀ is ctDNA MAF in the first sample and C₁ is ctDNA MAF in one of the additional samples. The MAF threshold, for example, can be, or be about, −0.005, −0.01, −0.015, −0.02, −0.025, −0.03, −0.035, −0.04, −0.045, −0.05, −0.055, −0.06, −0.065, −0.07, −0.075, −0.08, −0.085, −0.09, −0.095, −0.1, a range between any two of these values, or a value between −0.005 to −0.1. In some embodiments, the MAF threshold is at least, or at least about, −0.005, −0.01, −0.015, −0.02, −0.025, −0.03, −0.035, −0.04, −0.045, −0.05, −0.055, −0.06, −0.065, −0.07, −0.075, −0.08, −0.085, −0.09, −0.095, or −0.1. In some embodiments, the MAF threshold is −0.05. In some embodiments, the MAF threshold is −0.06.

The driver mutation can be, for example, a mutation in one of the 75 genes set forth in Table 3. In some embodiments, at least one of the one or more the driver mutations is a mutation in in the 75 genes set forth in Table 3. In some embodiments, one or more the driver mutations are mutations in the 75 genes set forth in Table 3. The driver mutation, or at least one of the one or more driver mutations, can be in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the driver mutation or at least one of the one or more driver mutations is one or more of the mutations set forth in Table 12 as HGVSc. In some embodiments, the method further comprises determining variant allele frequency (e.g., the VAF for one or more of the driver mutations disclosed herein) in ctDNA, PBMCs and/or BMMCs of the subject.

The type of the leukemia can vary. For example, the leukemia can be advanced, metastatic, refractory, and/or relapsed. In some embodiments, the leukemia is acute myeloid leukemia (AML), myelodysplastic syndrome (MDS) or chronic melomonocytic leukemia (CMML). In some embodiments, the leukemia is relapsed or refractory acute myeloid leukemia.

The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

The leukemia treatment can comprise standard of care therapies for leukemia. In some embodiments, the leukemia treatment comprises administration (for example, oral administration) of a Polo-like kinase 1 (PLK1) inhibitor (for example, onvansertib). The treatment can comprise administration of onvansertib for a desired duration in a cycle. The desired duration can be one, two, three, four, five, six, seven, eight, nine, ten, or more days. The cycle can be, for example, at least 20 days, 21 days, 22 days, 23 days, 24 days, 25 days, or more. For example, a single cycle of the treatment can comprise administration of onvansertib for four days, five days, six days, seven days, eight days, nine days, ten days, eleven days, twelve days, thirteen days, fourteen days, fifteen days, sixteen days, seventeen days, eighteen days, nineteen days, twenty days, or more in a cycle (e.g., a cycle of at least 21 days (e.g., 21 to 28 days)). In some embodiments, the treatment can comprise administration of onvansertib for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of onvansertib in a single cycle of the treatment can be continuous or with one or more intervals (e.g., one day or two days of break). In some embodiments, the treatment comprises administration of onvansertib for five days in a cycle of 21 to 28 days. The treatment can comprise administration of onvansertib at, or at about, 12 mg/m²-90 mg/m², for example, as a daily dose. For example, the treatment can comprise daily administration of onvansertib at, or at about, 8 mg/m², 10 mg/m², 12 mg/m², 14 mg/m², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a range between any two of these values, or any value between 8 mg/m²-90 mg/m². In some embodiments, the daily dose of onvansertib can be adjusted (e.g., increased or decreased with the range) during the treatment, or during a single cycle (e.g., the first cycle, the second cycle, the third cycle, and a subsequent cycle) of the treatment, for the subject.

A maximum concentration (Cₘₐₓ) of onvansertib in a blood of the subject (during the treatment or after the treatment) can be from about 100 nmol/L to about 1500 nmol/L. For example, the Cₘₐₓ of onvansertib in a blood of the subject can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of onvanserib in a blood of the subject over time can be from about 1000 nmol/L·hour to about 400000 nmol/L·hour. For example, the AUC of a plot of a concentration of onvansertib in a blood of the subject over time can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of onvansertib in a blood of the subject can be from about 1 hour to about 5 hours. For example, the time (Tₘₐₓ) to reach a maximum concentration of onvansertib in a blood of the subject can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of onvansertib in a blood of the subject can be from about 10 hours to about 60 hours. For example, the elimination half-life (T_{1/2}) of onvansertib in a blood of the subject can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

The leukemia treatment can comprise at least one additional administration of cancer therapeutics or cancer therapy. The PLK inhibitor and the cancer therapeutics or cancer therapy can, for example, co-administered simultaneously or sequentially. In some embodiments, the additional cancer therapeutics is cytarabine, low-dose cytarabine (LDAC) and/or decitabine. In some embodiments, the treatment comprises administration of LDAC at, or at about, 20 mg/m² subcutaneous (SC) once a day (qd) for seven, eight, night, ten, eleven, twelve, or thirteen days in a cycle. In some embodiments, the treatment comprises administration of decitabine at, or at about, 20 mg/m² intravenous (IV) qd for three, four, five, six, or seven days in a cycle. In some embodiments, the treatment comprises administration of LDAC at, or at about, 20 mg/m² subcutaneous (SC) once a day (qd) for ten days in a cycle, and administration of decitabine at 20 mg/m² intravenous (IV) qd for five days in a cycle.

The method can comprise analyzing ctDNA of the subject before the treatment. The treatment can comprise one or more cycles, and the ctDNA is analyzed before, during and after one or more cycles of the treatment. For example, the ctDNA can be analyzed before, during and after two or more cycle of the treatment, three or more cycle of the treatment, or each cycle of the treatment. Each cycle of treatment can be at least 21 days, for example, 21 days, 22 days, 23 days, 24 days, 25 days, 26 days, 27 days, 28 days, 29 days, 30 days, or more, or a range between any two of these values. In some embodiments, each cycle of treatment is from about 21 days to about 28 days. In some embodiments, each cycle of treatment is from 21 days to 28 days. In some embodiments, the subject is human.

Provided in some embodiments include kits comprising: a PLK1 inhibitor (e.g., onvansertib) or a pharmaceutically acceptable salt, solvate, stereoisomer thereof, and manual providing instructions for performing one or more of the steps for one or more methods disclosed herein. In some embodiments, the kit comprises: a PLK1 inhibitor (e.g., onvansertib) or a pharmaceutically acceptable salt, solvate, stereoisomer thereof, and manual providing instructions for performing one or more steps of the method disclosed herein for determining responsiveness of a subject to a leukemia treatment. For example, the method can comprise: analyzing circulating tumor DNA (ctDNA) of a subject with leukemia, wherein the subject is undergoing a treatment and/or has received a treatment for leukemia, thereby determining the responsiveness of the subject to the leukemia treatment. In some embodiments, analyzing ctDNA comprises detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, wherein a decrease in the variant allele frequency in at least one of the additional samples relative to the first sample indicates the subject as responsive to the leukemia treatment

In some embodiments, the kit comprises: a PLK1 inhibitor (e.g., onvansertib) or a pharmaceutically acceptable salt, solvate, stereoisomer thereof, and manual providing instructions for performing one or more steps of the method disclosed herein for determining leukemia status of a subject. The manual can be, for example, a package insert for a pharmaceutical product, or a label for a pharmaceutical product. For example, the method can comprise: analyzing circulating tumor DNA (ctDNA) of a subject, wherein the subject is undergoing a current treatment for leukemia, has received a prior treatment for leukemia, and/or is in remission for leukemia, thereby determining leukemia status of the subject. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, wherein an increase in the variant allele frequency at the additional sample(s) relative to the first sample indicates that the subject is at risk of leukemia relapse or is in leukemia relapse.

In some embodiments, the kit comprises: a PLK1 inhibitor (e.g., onvansertib) or a pharmaceutically acceptable salt, solvate, stereoisomer thereof, and manual providing instructions for performing one or more steps of the method disclosed herein for improving treatment outcome for leukemia. For example, the method can comprise: detecting variant allele frequency in circulating tumor DNA (ctDNA) obtained from a subject at a first time point in a first sample before the subject undergoes a leukemia treatment; detecting variant allele frequency in ctDNA obtained from the subject at one or more additional time points in one or more additional samples after the subject undergoes the leukemia treatment; determining the difference of the variant allele frequency in ctDNA between the first and at least one of the one or more additional samples, wherein a decrease in the variant allele frequency in at least one of the additional samples relative to the first sample indicates the subject as responsive to the leukemia treatment; and continuing the leukemia treatment to the subject if the subject is indicated as responsive to the leukemia treatment, or discontinuing the leukemia treatment to the subject and/or starting a different leukemia treatment to the subject if the subject is not indicated as responsive to the leukemia treatment.

In some embodiments, the kit comprises: a PLK1 inhibitor (e.g., onvansertib) or a pharmaceutically acceptable salt, solvate, stereoisomer thereof, and manual providing instructions for performing one or more steps of the method disclosed herein for treating leukemia. For example, the method can comprise: administering a leukemia treatment to a subject in need thereof; determining a decrease, relative to a variant allele frequency in a first sample of the subject obtained at a first time point before the subject receives the leukemia treatment, in a variant allele frequency in a second sample of the subject obtained at a second time point after the subject receives the leukemia treatment; and continuing with the leukemia treatment.

### EXAMPLES

Some aspects of the embodiments discussed above are disclosed in further detail in the following examples, which are not in any way intended to limit the scope of the present disclosure.

### Example 1

### Plasma-Derived ctDNA as a Surrogate Biomarker for Treatment Response with Onvansertib, in Combination with LDAC or Decitabine in Acute Myeloid Leukemia (AML)

This example describes results from a Phase 1b/2 clinical trial, having ClinicalTrials.gov Identifier NCT03303339, and entitled “Onvansertib in Combination With Either Low-dose Cytarabine or Decitabine in Adult Patients With Acute Myeloid Leukemia (AML).” In the study, a PLK1 inhibitor, onvansertib, was used in combination with either low-dose cytarabine (LDAC) or decitabine in patients with relapsed or refractory (R/R) AML.

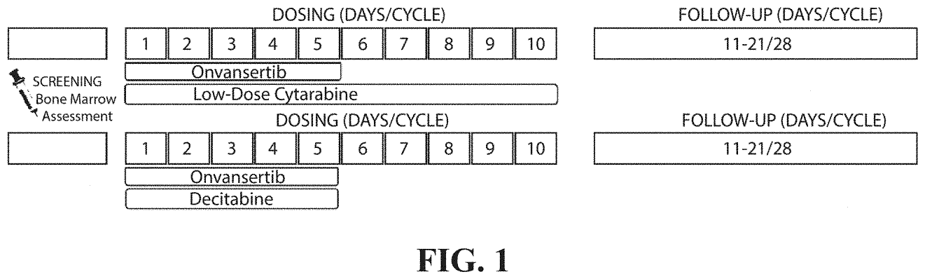

Treatment plan and study design: Onvansertib was administered orally, in escalating doses, on days 1 through 5 in combination with either LDAC (arm A, 20 mg/m² subcutaneously once daily on days 1 through 10) or decitabine (arm B, 20 mg/m² intravenously over 1 hour on days 1 through 5) in a 28-day cycle. Investigators had the flexibility to shorten the cycle to 21 days if they judged that more frequent dosing could benefit the patient. A non-limiting illustration of the dosing regime is shown in FIG. 1. The primary objectives were to evaluate dose-limiting toxicities (DLTs) and the maximum tolerated dose (MTD) or recommended phase 2 dose (RP2D) of onvansertib. The first dose of onvansertib was 12 mg/m², which was half of RP2D established in the phase I single-agent study for solid tumors. Each arm followed a standard 3+3 dose-escalation design, in which onvansertib dose was escalated by 50% increments. Three patients were treated, and if there were no DLTs in the first cycle, escalation to the next higher dose occurred. If a DLT was reported, an additional 3 patients were treated at that dose. If ≥2 patients experienced a DLT, the dose was considered nontolerated, and lower doses were explored in subsequent cohorts. Subjects who had not received at least 80% of the dose of study drug(s) during the first cycle or who discontinued for any reason other than DLT were replaced. The MTD was defined as the highest dose achieved at which no more than one of six subjects experienced a DLT. The RP2D was determined based on the assessment of safety, pharmacokinetics, and preliminary efficacy in subjects treated at a dose cleared for safety. For the study, the primary endpoint was to evaluate first-cycle dose-limiting toxicities and the MTD. Secondary and exploratory endpoints included safety, pharmacokinetics, antileukemic activity, and response biomarkers.

Study Primary Objectives: Phase 1b: Assess safety, define dose-limiting toxicities (DLTs) and MTD/RP2D. Phase 2: Assess safety, tolerability and preliminary anti-leukemic activity at the MTD (or RP2D).

Patient selection: Patients 18 years or older with a confirmed diagnosis of AML and Eastern Cooperative Oncology Group (ECOG) performance status of 0-2 were eligible. Patients were allowed up to three prior treatment regimens for their AML, including HMAs. Induction therapy and hematopoietic stem cell transplant were counted as one prior line of therapy. Treatment-naïve patients who were unfit for intensive induction therapy were also eligible. Treatments for preexisting myelodysplastic syndrome (MDS) were allowed and not considered as prior treatment. Patients with treatment-related AML and acute promyelocytic leukemia were excluded, as were patients with aspartate aminotransferase and/or alanine aminotransferase ≥2.5×upper limit of normal, total bilirubin ≥2 mg/dL, or serum creatinine ≥2.0 mg/dL. The protocol was approved by the institutional review board or independent ethics committees at each participating center and was in accordance with the Declaration of Helsinki and Good Clinical Practice guidelines. Written informed consent was obtained from all patients before screening.

Safety: Patients were evaluable for safety if they had received at least one dose of onvansertib. Safety evaluations included physical examinations, laboratory test results, electrocardiograms, and monitoring of adverse events (AE) graded by the NCI Common Terminology Criteria for Adverse Events (NCI CTCAE version 4.03). Investigators assessed causality as either unrelated, unlikely, possibly, probably, or definitely related to study drugs. DLTs (defined in Table 1) were evaluated during the first cycle of treatment.

TABLE 1
Definition of dose-limiting toxicities (DLTs).
The following first-cycle adverse events (AEs)
met the definition of DLT if considered by
the investigator as possibly related to treatment
with onvansertib in combination with chemotherapy

| | |
|---|---|
| Non- | Grade 3 AEs that were clinically significant |
| hematological | and persisted >7 days without decreasing |
| AEs | in severity despite standard of care |
| | Grade 4 AEs that were symptomatic |
| Hematological | Persistent pancytopenia resistant |
| AEs | to current standard of care that |
| | continued for >42 days, not related to |
| | leukemic infiltration or considered related |
| | to study therapy. |

Antileukemic activity: Bone marrow evaluations were performed at screening, between days 15-28 of cycles 1 and 2, and following every other subsequent cycle if considered clinically appropriate by the investigator. Response to treatment was evaluated by the investigator using the modified International Working Group criteria 2003, detailed in Table 2. Antileukemic activity was assessed in all patients evaluable for DLTs.

TABLE 2
Response to treatment based on the modified International
Working Group criteria 2003.

| | |
|---|---|
| Complete remission (CR) | Bone marrow blast <5%, |
| | neutrophils ≥1 × 109/L, platelets |
| | ≥100 × 109/L |
| CR with incomplete | All CR criteria except for residual |
| hematologic recovery (CRi) | neutropenia and thrombocytopenia |
| Morphologic leukemia- | Bone marrow blasts <5%, no |
| free state (MLFS) | hematological recovery |
| Partial remission (PR) | All hematologic criteria of CR, |
| | decrease in bone marrow blasts |
| | by at least 50% with blasts |
| | between 5% and 25% |

Pharmacokinetics: Blood samples were collected from patients for pharmacokinetic analysis on days 1 and 5 at predose; at 0.5, 1, 2, 3, 4, 8, and 24 hours after administration of onvansertib; and once on days 8, 15, and 22. Onvansertib plasma concentrations were determined by LC/MS-MS at PRA Bioanalytical Laboratory. The pharmacokinetics parameters, including maximum plasma concentration (Cₘₐₓ), time to maximum plasma concentration (tₘₐₓ), half-life (t_{1/2}), and AUC, were calculated for each patient using Phoenix WinNonlin.

Correlative Studies:

Plasma inhibitory activity assay: Onvansertib plasma inhibitory activity was assessed by measuring changes in the phosphorylation of the PLK1 substrate, TCTP, as described previously for FLT3 inhibitors. HL-60 cell line used for this assay was obtained from the ATCC and cultured in Iscove's modified Dulbecco's medium supplemented with 20% FBS. The cell line was not tested for Mycoplasma. Briefly, 3×106 HL-60 cells (passage 2-5) were incubated with 1 mL of patient plasma at 37° C. for 1 hour. Cells were washed, lysed, and changes in phosphorylated TCTP (pTCTP)/TCTP were assessed by immunoblot. Plasma samples from patients treated with onvansertib (12-60 mg/m²) in combination with LDAC or decitabine were used. For each dose level, 3-4 patients were analyzed (a total of 18 patients) on the basis of the availability of plasma samples, and each dose level included at least 1 patient of each arm (LDAC/decitabine). For each patient, pTCTP/TCTP level was quantified in cells incubated with plasma collected on day 1 at predose and postdose (tₘₐₓ) and normalized to day 1 predose sample to measure the % pTCTP inhibition by onvansertib-containing plasma.

Blood collection and processing for correlative studies: Blood samples were collected from patients into CellSave Preservative Tubes (Silicon Biosystems) and EDTA tubes on days 1 (predose and 3 hours postdose), 5, 8, 15, and 22 of each cycle and processed 24 hours after collection at Cardiff Oncology's laboratories. Plasma was separated from whole blood collected in CellSave tube by centrifugation. Peripheral blood mononuclear cells (PBMC) and bone marrow mononuclear cells (BMMC) were isolated by density gradient centrifugation over Histopaque-1077 (Sigma Aldrich), following the manufacturer's instructions. To quantify AML blasts, cells were labeled with a cocktail consisting of fluorochrome-labeled antibodies to CD45, CD34, CD117, CD13, and HLA-DR (BioLegend). Samples were analyzed with a FACSCanto II (BD Biosciences). AML blasts were identified on the basis of a low side scatter/CD45dim profile and expression of blast markers. Genomic DNA (gDNA) was extracted from PBMCs and BMMCs using the QIAamp DNA Blood Kit (Qiagen) and circulating tumor DNA (ctDNA) from plasma using QIAamp Circulating Nucleic Acid Kit (Qiagen) according to the manufacturer's recommendations. DNA was quantified with a Qubit 3.0 Fluorometer (Thermo Fisher Scientific). Targeted next-generation sequencing (NGS) was performed with the VariantPlex Myeloid Panel (ArcherDx), which includes 75 genes known to be associated with AML (Table 3).

TABLE 3
VariantPlex myeloid panel.

| | | | | |
|---|---|---|---|---|
| ABL1 | CEBPA | GNAS | MYD88 | SF3B1 |
| ANKRD26 | CSF3R | HRAS | NF1 | SH2B3 |
| ASXL1 | CUXI | IDH1 | NOTCH1 | SLC29A1 |
| ATRX | CXCR4 | IDH2 | NPM1 | SMCIA |
| BCOR | DCK | IKZF1 | NRAS | SMC3 |
| BCORL1 | DDX41 | JAK2 | PDGFRA | SRSF2 |
| BRAF | DHX15 | JAK3 | PHF6 | STAG2 |
| BTK | DNMT3A | KDM6A | PPMID | STAT3 |
| CALR | ETNK1 | KIT | PTEN | TET2 |
| CBL | ETV6 | KMT2A | PTPN11 | TP53 |
| CBLB | EZH2 | KRAS | RAD21 | U2AF1 |
| CBLC | FBXW7 | LUC7L2 | RBBP6 | U2AF2 |
| CCND2 | FLT3 | MAP2K1 | RPS14 | WT1 |
| CDC25C | GATA1 | MPL | RUNX1 | XPO1 |
| CDKN2A | GATA2 | MYC | SETBP1 | ZRSR2 |

NGS libraries were sequenced on the MiSeq Platform (Illumina) with MiSeq reagent kit v3. Data analysis was performed using Archer Analysis version 6.0.3.2 (ArcherDX) with custom targets, VariantPlex_Myeloid_GSP5031-v1.0, and targeted mutations, Archer Comprehensive Targets v1.1. For 20 patients, a driver mutation was identified by targeted NGS, and probe-based assays enabling detection of variant and wild-type alleles were commercially obtained (PrimePCR, Bio-Rad Laboratories, or TaqMan SNP Genotyping Assays, Thermo Fisher Scientific). Digital droplet PCR (ddPCR) was performed using the Bio-Rad QX200 Droplet Digital PCR System following the manufacturer's protocols, targeting an identified driver mutation, for enumerating mutant allele frequencies in Plasma (ctDNA), PBMCs, and BMMCs. Each sample was analyzed using at least two technical replicates. A Poisson correction was applied for independent enumeration of mutant alleles and wild-type alleles to calculate mutant allele frequency (MAF). Data analysis was carried out using the QuantaSoft Software, version 1.7 (Bio-Rad).

Protein extract preparation and immunoblot: Protein extracts were prepared from PBMCs isolated from CellSave tube and from HL-60 cells using M-PER buffer with Protease and Phosphatase Inhibitor Cocktails (Thermo Fisher Scientific). Protein concentration was measured with the Pierce BCA Protein Assay Kit (Thermo Fisher Scientific). Western blots were performed as Simple Western Assays using the Wes System (ProteinSimple), a combination of capillary electrophoresis and immunodetection techniques, following the manufacturer's protocols. Primary antibodies were purchased from Cell Signaling Technology: phospho-TCTP-Ser46 (#5251) and TCTP (#5128). Quantitative analysis was performed using Compass Software (ProteinSimple). Signal intensity (area) of pTCTP was normalized to the peak area of TCTP and reported as pTCTP/TCTP or % pTCTP.

Statistical analysis: Pearson correlation was used to analyze the relationship between plasma ctDNA MAF and bone marrow MAF. A Fisher exact test was performed to test the association between target engagement and decrease in bone marrow blasts. The AUC of the ROC curve was used to measure the performance of the log₂(C₁/C₀) to predict clinical response. The ROC curve was used to select the optimal threshold for prediction based on the Youden method and the sensitivity and specificity at this threshold were estimated. Ninety-five percent confidence intervals (CI) were calculated for the AUC, and for all parameters at the threshold, using 2,000 stratified bootstrap replicates. All statistical tests were performed using R version 4.0.2 and GraphPad Prism version 8.4.3.

Patients Characteristics: A total of 40 patients were enrolled in this phase Ib study in nine centers in the U.S. between January 2018 and August 2019. They were treated in two noncomparative study arms: arm A (onvansertib at 12-90 mg/m²+LDAC, n=17) and arm B (onvansertib at 12-90 mg/m²+decitabine, n=23). Patients were assigned to either arm on the basis of investigator discretion and slot availability. At data cutoff, Oct. 31, 2019, 5 patients continued to receive onvansertib on-study: 4 patients in the decitabine arm and 1 patient in the LDAC arm. Baseline characteristics are summarized in Table 4 and detailed in Table 5. The median age was 68 years (range, 33-88), and 70% of patients were male. Twenty-seven (68%) patients had adverse risk cytogenetics based on the 2017 ELN recommendations. At study entry, 20 (50%) patients had received one prior regimen for AML and 15 (38%) patients had received two or three prior treatments. There were 5 (12.5%) patients with untreated AML, but four of them had received HMAs for a previous diagnosis of MDS. The median proportion of bone marrow blasts was 26% (range, 5-95) with 55% of patients having >20% bone marrow blasts at baseline. Two (9%) patients in the decitabine arm had prior decitabine treatment and 8 (47%) patients in the LDAC arm had prior cytarabine treatment.

TABLE 4
Summary of baseline characteristics for
all patients enrolled.

| | Patients (N = 40) |
|---|---|
| Age, median (range) | 68 (33-88) |
| Male/female | 28/12 (70%/30%) |
| ECOG performance status | |
| 0 | 4 (10%) |
| 1 | 33 (82.5%) |
| 2 | 3 (7.5%) |
| Prior AML therapies | |
| 0 | 5 (12.5%) |
| 1 | 20 (50%) |
| 2+ | 15 (37.5%) |
| Prior cytarabine treatment (all patients) | 24 (60%) |
| LDAC arm (n = 17) | 8 (47%) |
| Decitabine arm (n = 23) | 16 (70%) |
| Prior decitabine treatment (all patients) | 10 (25%) |
| LDAC arm (n = 17) | 8 (47%) |
| Decitabine arm (n = 23) | 2 (9%) |
| Cytogenetic risk | |
| Favorable | 2 (5%) |
| Intermediate | 11 (27.5%) |
| Adverse | 27 (67.5%) |
| Percent bone marrow blasts, | 26 (5-95) |
| median (range) | |

TABLE 5
Baseline characteristics for all patients enrolled.

| | | | | Prior | Prior | Prior | | % bone |
|---|---|---|---|---|---|---|---|---|
| Patient | | | | AML | cytarabine | decitabine | Cytogenetic | marrow |
| ID | Age | Gender | ECOG | therapies | treatment | treatment | risk | blast |
| 01-002 | 88 | Male | 1 | 1 | No | Yes | Adverse | 25 |
| 01-021 | 77 | Male | 1 | 1 | No | No | Intermediate | 60 |
| 01-024 | 82 | Male | 1 | 2 | No | Yes | Intermediate | 14 |
| 03-015 | 60 | Male | 1 | 3 | Yes | Yes | Adverse | NA |
| 03-037 | 83 | Male | 0 | 1 | Yes | Yes | Intermediate | 10 |
| 03-051 | 68 | Male | 1 | 1 | Yes | No | Adverse | 12 |
| 03-056 | 88 | Male | 1 | 1 | No | No | Adverse | 35 |
| 03-060 | 66 | Female | 0 | 2 | No | Yes | Adverse | 21 |
| 05-016 | 53 | Male | 1 | 2 | Yes | Yes | Adverse | 5 |
| 05-030 | 68 | Female | 1 | 0 | No | No | Intermediate | 20 |
| 05-043 | 66 | Male | 1 | 2 | Yes | No | Intermediate | 19 |
| 07-004 | 73 | Male | 1 | 0 | No | No | Adverse | NA |
| 07-008 | 48 | Female | 1 | 1 | Yes | No | Adverse | 76 |
| 07-009 | 75 | Male | 1 | 1 | Yes | No | Adverse | 94 |
| 07-010 | 88 | Male | 1 | 1 | No | No | Adverse | 40 |
| 07-011 | 62 | Male | 1 | 1 | No | No | Adverse | 20 |
| 07-013 | 33 | Male | 1 | 1 | Yes | No | Intermediate | 27 |
| 07-018 | 65 | Female | 2 | 1 | Yes | No | Adverse | 7 |
| 07-033 | 49 | Male | 1 | 1 | Yes | No | Adverse | 66 |
| 07-035 | 76 | Male | 1 | 1 | Yes | No | Adverse | 20 |
| 07-036 | 75 | Female | 1 | 0 | No | No | Adverse | 40 |
| 07-038 | 63 | Male | 1 | 3 | Yes | No | Adverse | 83 |
| 07-059 | 88 | Female | 1 | 1 | No | No | Adverse | 15 |
| 07-062 | 70 | Male | 0 | 1 | Yes | No | Intermediate | 20 |
| 07-063 | 58 | Male | 1 | 1 | Yes | Yes | Adverse | 40 |
| 08-027 | 63 | Female | 2 | 2 | Yes | Yes | Adverse | 86 |
| 08-050 | 64 | Male | 1 | 2 | Yes | No | Intermediate | 82 |
| 08-055 | 66 | Male | 2 | 2 | Yes | Yes | Adverse | 6 |
| 08-058 | 51 | Female | 1 | 1 | Yes | No | Adverse | 11 |
| 08-061 | 67 | Female | 1 | 2 | Yes | No | Favorable | 57 |
| 09-026 | 64 | Male | 1 | 1 | Yes | No | Adverse | 20 |
| 09-032 | 73 | Male | 1 | 2 | Yes | Yes | Adverse | 66 |
| 09-034 | 76 | Male | 1 | 0 | No | No | Intermediate | 49 |
| 09-054 | 85 | Male | 1 | 2 | No | No | Adverse | 7 |
| 09-064 | 76 | Male | 1 | 0 | No | No | Intermediate | 15 |
| 11-019 | 68 | Female | 0 | 2 | Yes | No | Intermediate | 10 |
| 11-040 | 58 | Male | 1 | 1 | Yes | No | Favorable | 28 |
| 12-041 | 77 | Male | 1 | 1 | No | No | Adverse | 42 |
| 12-045 | 80 | Female | 1 | 3 | No | No | Adverse | 95 |
| 12-048 | 60 | Female | 1 | 2 | Yes | No | Adverse | 27 |

Onvansertib was well tolerated with most grades 3 and 4 adverse events related to myelosuppression. In the decitabine arm, the MTD was established at 60 mg/m², and 5 (24%) of the 21 evaluable patients achieved complete remission with or without hematologic count recovery. Patients were analyzed by targeted NGS, and the median number of genes with variants identified was 3 [range 1-7]. Among them, 20 patients were chosen for subsequent ctDNA analysis. Clinical response was evaluated by patient's pathologist using bone marrow aspirate (Table 6). Responders were defined as patients achieving complete response (BM blasts <5%) with or without count recovery (CR+CRi), and six patients were identified as responders (Table 7). As shown herein, decrease in mutant ctDNA during the first cycle of therapy was associated with clinical response. Engagement of the PLK1 target, TCTP, was measured in circulating blasts and was associated with greater decrease in bone marrow blasts.

TABLE 6
Results of clinical response evaluation

| Cytogenetic Risk | N | (%) |
|---|---|---|
| Favorable | 2 | 10% |
| Intermediate | 6 | 30% |
| Adverse | 9 | 45% |
| Unknown | 3 | 15% |

TABLE 7
Identification of responders using
ctDNA analysis

| | N | (%) |
|---|---|---|
| Responders | 6 | 30% |
| Non-Responders | 14 | 70% |

Dose escalation and toxicity. Onvansertib was investigated at five dose levels (12-60 mg/m²) in arm A (LDAC) and at six dose levels (12-90 mg/m²) in arm B (decitabine). At data cutoff, the median number of completed onvansertib cycles for both arms was 2 (range, 0-18), and the median time on treatment was 53 days (range, 11-574). Five patients (1 in arm A and 4 in arm B) had at least one cycle of less than 28 days (24 or 25 days). Treatment with onvansertib was well tolerated in both arms through the first five dose levels (12-60 mg/m²), with no DLTs reported. In arm A, no higher dose levels were explored. In arm B, 2 of 6 patients treated at dose level 6 (90 mg/m²) experienced a DLT during the first cycle of treatment, consisting of grade 3 (G3) mucositis and grade 4 (G4) rash, respectively. Subsequently, onvansertib MTD in combination with decitabine was established at 60 mg/m².

Treatment-emergent AEs reported in at least 10% of patients are listed in Table 8. The most common G3 and G4 AEs were anemia (35%), febrile neutropenia (30%), neutropenia (25%), thrombocytopenia (25%), leukopenia (12.5%), and stomatitis (12.5%). G3 and G4 AEs considered possibly related to onvansertib were mostly hematologic: neutropenia (15%), anemia (12.5%), thrombocytopenia (12.5%), and leukopenia (5%). G3 stomatitis, reported in 3 patients (8%) at the highest dose levels (one at 60 mg/m² and two at 90 mg/m²), was the only nonhematologic G3/G4 AE possibly related to onvansertib reported in ≥5% patients. Two patients (one from 18 mg/m² and one from 27 mg/m² dose level) had a dose reduction of onvansertib (dose level −1) due to hematologic toxicities. Three patients were discontinued from therapy because of treatment-related AEs, including grade 2 (G2) fatigue, G3 mucositis (DLT), and G4 rash (DLT).

TABLE 8
Treatment-emergent AEs reported in ≥10% of all patients (N = 40).

| AE | Grade 1-2 (%) | Grace 3-4 (%) | All grades (%) |
|---|---|---|---|
| Anemia | 1 (2.5) | 14 (35) | 15 (37.5) |
| Fatigue | 14 (35) | | 14 (35) |
| Febrile neutropenia | | 12 (30) | 12 (30) |
| Nausea/vomiting | 12 (30) | | 12 (30) |
| Thrombocytopenia | 2 (5) | 10 (25) | 12 (30) |
| Rash/pruritus | 10 (25) | 1 (2.5) | 11 (27.5) |
| Dyspnea | 8 (20) | 2 (5) | 10 (25) |
| Neutropenia | | 10 (25) | 10 (25) |
| Stomatitis | 4 (10) | 5 (12.5) | 9 (22.5) |
| Diarrhea | 8 (20) | | 8 (20) |
| Edema | 6 (15) | 2 (5) | 8 (20) |
| Constipation | 6 (15) | | 6 (15) |
| Cough | 6 (15) | | 6 (15) |
| Decreased appetite | 6 (15) | | 6 (15) |
| Epistaxis | 6 (15) | | 6 (15) |
| Dizziness | 5 (12.5) | | 5 (12.5) |
| Pyrexia | 5 (12.5) | | 5 (12.5) |
| Leukopenia | | 5 (12.5) | 5 (12.5) |
| Blood bilirubin increased | 3 (7.5) | 1 (2.5) | 4 (10) |
| Headache | 4 (10) | | 4 (10) |
| Lung infection | | 4 (10) | 4 (10) |
| Oropharyngeal pain | 4 (10) | | 4 (10) |

There were a total of 71 serious AEs (SAE) reported in 33 patients (Table 9). The most common SAEs were: febrile neutropenia (25 events in 17 patients), lung infection (5), and pneumonia (4). Nine (13%) SAEs considered as possibly related to onvansertib consisted of G3 febrile neutropenia (n=1, resolving within 1 week), G4 neutropenia (n=1), G3 stomatitis (n=1), G2 rash maculopapular (n=1), G3 palmar-plantar erythrodysesthesia syndrome (n=1), and G4 rash (n=1). Three deaths occurred while on treatment, all of which were related to AML or its complications: 2 patients died because of progressive disease and 1 from intracranial hemorrhage after a fall.

TABLE 9
Serious adverse events.

| | Number | Number |
|---|---|---|
| Serious adverse events | of events | of patients |
| Febrile neutropenia | 25 | 17 |
| Lung infection | 5 | 5 |
| Pneumonia | 3 | 3 |
| Pyrexia | 2 | 2 |
| Aphasia | 1 | 1 |
| Appendicitis | 1 | 1 |
| Arthralgia | 1 | 1 |
| Asthenia | 1 | 1 |
| Atrial fibrillation | 1 | 1 |
| Back pain | 1 | 1 |
| Disease progression | 1 | 1 |
| Dyspnea | 1 | 1 |
| Face edema | 1 | 1 |
| Hematuria | 1 | 1 |
| Hemorrhage intracranial | 1 | 1 |
| Hyponatremia | 1 | 1 |
| Infusion related reaction | 1 | 1 |
| Leukemic infiltration | 1 | 1 |
| Lower gastrointestinal | 1 | 1 |
| hemorrhage | | |
| Lung consolidation | 1 | 1 |
| Neck pain | 1 | 1 |
| Neutrophil count decreased | 1 | 1 |
| Edema peripheral | 1 | 1 |
| Pain in jaw | 1 | 1 |
| Palmar-plantar | 1 | 1 |
| erythrodysaesthesia syndrome | | |
| Pharyngeal edema | 1 | 1 |
| Physical deconditioning | 1 | 1 |
| Pleural effusion | 1 | 1 |
| Pneumonia fungal | 1 | 1 |
| Pulmonary mass | 1 | 1 |
| Rash maculo-papular | 1 | 1 |
| Septic shock | 1 | 1 |
| Seroma | 1 | 1 |
| Skin lesion | 1 | 1 |
| Soft tissue infection | 1 | 1 |
| Staphylococcal bacteremia | 1 | 1 |
| Stomatitis | 1 | 1 |
| Tumor lysis syndrome | 1 | 1 |
| Urine output decreased | 1 | 1 |

Pharmacokinetics: Table 10 shows mean pharmacokinetics parameters on day 5 of cycle 1 for all patients enrolled. The Cₘₐₓ and AUC from 0 to 24 hours (AUC₀₋₂₄) increased proportionally with the administrated dose in both arms. The tₘₐₓ was achieved between 1.7 and 3.3 hours and the median half-life of onvansertib (t_{1/2}) was 26.4 hours (range, 16-46.5), and these parameters were independent of the administrated dose or combination treatment. Overall, pharmacokinetics parameters were similar with either LDAC or decitabine as those reported for onvansertib single agent in the phase I solid tumor study.

TABLE 10
Cycle 1 day 5 mean ± SD pharmacokinetic parameters of onvansertib.

| Dose | Combination | Number of | tₘₐₓ | Cₘₐₓ | AUC₍₀₋₂₄₎ | t_{1/2} |
|---|---|---|---|---|---|---|
| (mg/m²) | treatment | patients | (hour) | (nmol/L) | (nmol/L · hour) | (hour) |
| 12 | Decitabine | 4 | 2.5 ± 0.6 | 163 ± 90 | 2,270 ± 1,440 | 26 ± 16 |
| 12 | LDAC | 3 | 2.0 ± 1.0 | 153 ± 84 | 2,150 ± 833 | 32 ± 21 |
| 18 | Decitabine | 3 | 2.7 ± 0.6 | 230 ± 129 | 3,380 ± 1,740 | 21 ± 7 |
| 18 | LDAC | 3 | 3.0 ± 1.0 | 109 ± 39 | 1,730 ± 847 | 33 ± 18 |
| 27 | Decitabine | 3 | 2.0 ± 1.7 | 4112 ± 118 | 5,420 ± 402 | 16 ± 6 |
| 27 | LDAC | 3 | 2.7 ± 1.5 | 340 ± 219 | 4,470 ± 1,450 | 18 ± 5 |
| 40 | Decitabine | 4 | 3.3 ± 1.0 | 539 ± 228 | 8,050 ± 3,380 | 25 ± 15 |
| 40 | LDAC | 3 | 1.7 ± 1.2 | 350 ± 104 | 4,270 ± 1,950 | 16 ± 7 |
| 60 | Decitabine | 3 | 3.3 ± 1.2 | 1,040 ± 534 | 16,300 ± 6,690 | 47 ± 8 |
| 60 | LDAC | 5 | 2.4 ± 1.1 | 905 ± 330 | 12,700 ± 4,850 | 37 ± 13 |
| 90 | Decitabine | 6 | 3.3. ± 0.8 | 1,310 ± 806 | 21.800 ± 18,400 | 30 ± 11 |

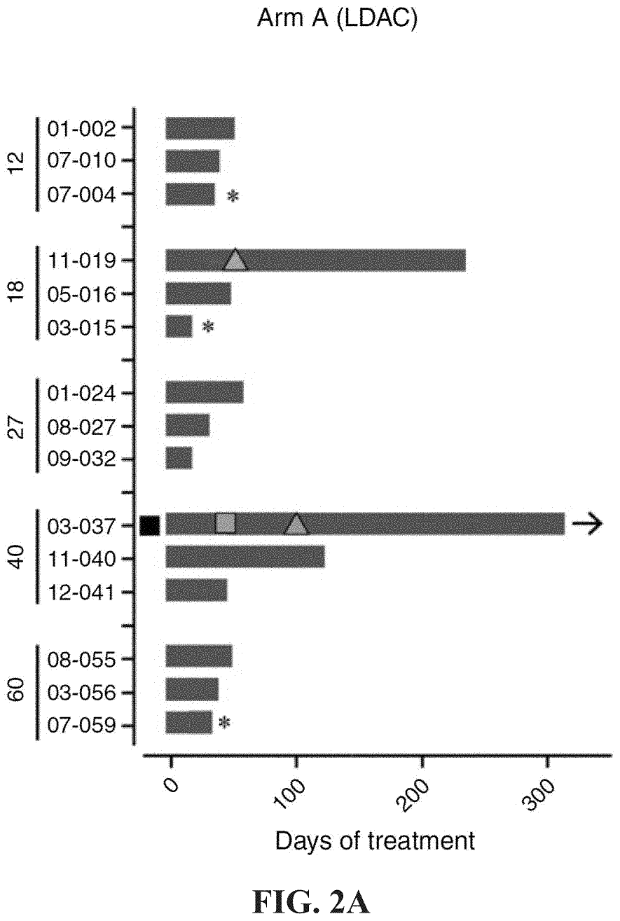

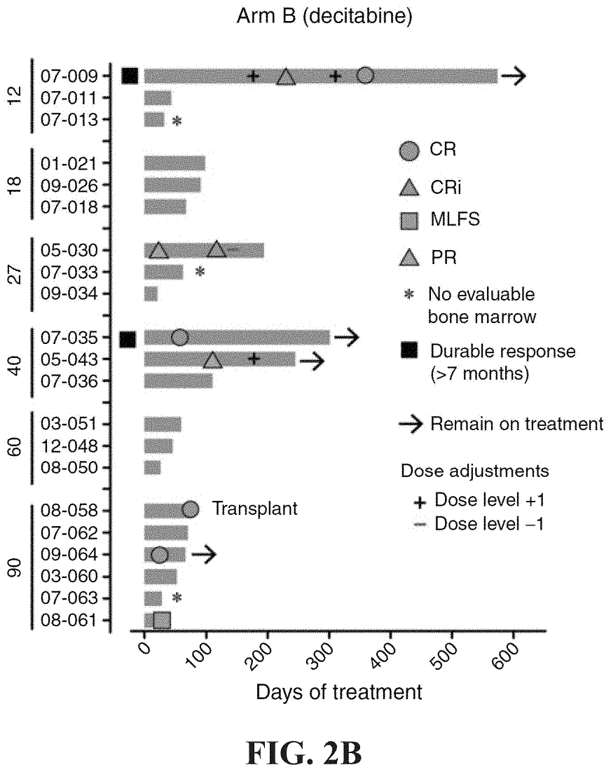

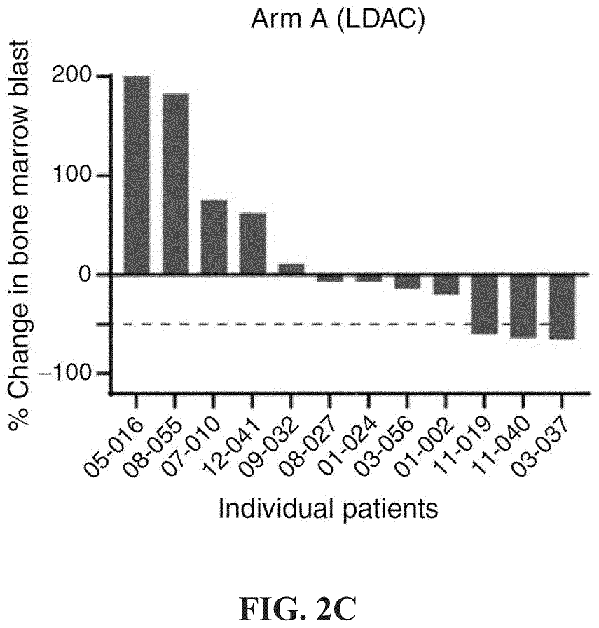

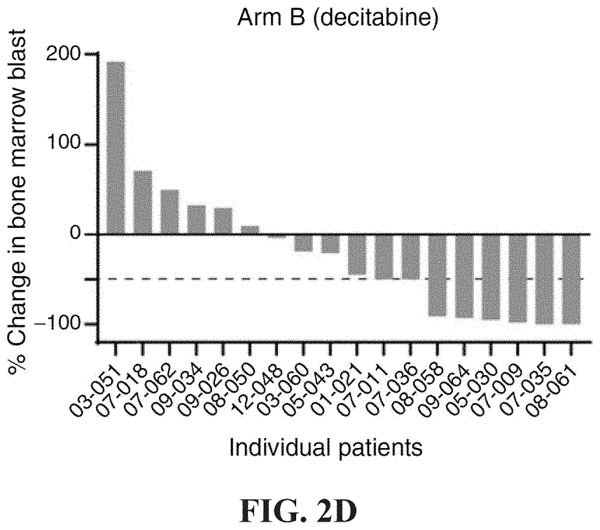

Clinical response and duration: Swimmer plots are shown for patients evaluable for efficacy (FIGS. 2A-B). In the decitabine arm, 5 (24%) patients achieved complete remission with or without incomplete hematopoietic recovery (CR/CRi), 4 patients had a CR and 1 a CRi. In the LDAC arm, 1 (7%) of the 15 patients achieved CRi. Overall response rate (ORR), including CR, CRi, morphologic leukemia free-state (MLFS), and partial response (PR), was 33% (7/21 patients) in the decitabine arm and 13% (2/15 patients) in the LDAC arm. Eight (44%) of the 18 patients with evaluable bone marrow biopsy showed a ≥50% reduction in blasts in the decitabine arm and 3 (25%) of 12 patients in the LDAC arm (FIGS. 2C-D).

In the decitabine arm, CR/CRi was documented with onvansertib at doses of 27 (n=2), 40 (n=1), and 90 mg/m² (n=2). The median number of cycles to achieve CR/CRi was 4 (range, 1-7) and 1 patient proceeded to transplant immediately after achieving CR. The median duration of CR/CRi was 5.5 months (range, 1.5-11.5). Three of the 5 responders remained on treatment at the data cutoff, with a time since CR/CRi of 1.5, 8, and 11.5 months, respectively. Two responders discontinued treatment: 1 patient proceeded to transplant and 1 patient progressed 2.5 months after remission. Responders had a median age of 75 years (range, 51-76). Two patients were treatment naïve for their AML, but had received azacitidine for MDS, and 3 patients had either relapsed (n=2) from or were refractory (n=1) to prior induction chemotherapy.

Molecular Profiling: A targeted NGS assay covering 75 genes was used to identify driver mutations. A droplet digital PCR (ddPCR) assay (Bio-Rad) targeting an identified driver mutation was used to enumerate mutant allele frequencies in plasma (ctDNA), PBMCs, and BMMCs.

ctDNA as a Predictive Biomarker for Treatment Response

Mutational profiling was performed at baseline for all patients (n=40) using DNA from PBMCs or BMMCs. The most frequently mutated genes were TP53 (28%), ASXL1 (20%), SRSF2 (18%), NRAS (18%), DNMT3A (18%), TET2 (18%), FLT3 (16%), and SF3B1 (10%; Tables 11 and 12). Patients achieving CR/CRi (n=6) had mutations in splicing factors (n=4), signaling effectors (n=3), and DNA methylation-related genes (n=2; Tables 12 and 13).

TABLE 11
Summary of mutational analysis
Mutational profiling (n = 40)

| Gene | Patients (n) | Patients (%) |
|---|---|---|
| TP53 | 11 | 28% |
| ASXL1 | 8 | 20% |
| DNMT3A | 7 | 18% |
| NRAS | 7 | 18% |
| SRSF2 | 7 | 18% |
| TET2 | 7 | 18% |
| SF3B1 | 4 | 10% |
| FLT3 | 3 | 8% |
| FLT3 ITD | 3 | 8% |
| IDH2 | 3 | 8% |
| NPM1 | 3 | 8% |
| RUNX1 | 3 | 8% |
| CDKN2A | 2 | 5% |
| KRAS | 2 | 5% |
| STAG2 | 2 | 5% |
| CALR | 1 | 3% |
| CBL | 1 | 3% |
| CSF3R | 1 | 3% |
| DDX41 | 1 | 3% |
| GATA2 | 1 | 3% |
| JAK2 | 1 | 3% |
| PHF6 | 1 | 3% |
| SETBP1 | 1 | 3% |

TABLE 12
Detailed list of variants identified by targeted-NGS

| | | | Allelic | COSMIC |
|---|---|---|---|---|
| Gene | HGVSp | HGVSc | Frequency | ID |
| SRSF2 | NP_001182356.1:p. | NM_001195427.1:c.284C > G | 0.1365 | COSM211661 |
| | Pro95Arg | | | |
| STAG2 | NP_001036214.1:p. | NM_001042749.1:c.1908C > G | 0.2597 | COSM3405922 |
| | Tyr636Ter | | | |
| SRSF2 | NP_001182356.1:p. | NM_001195427.1:c.284C > G | 0.2603 | COSM211661 |
| | Pro95Arg | | | |
| STAG2 | NP_001036214.1:p. | NM_001042749.1:c.1908C > G | 0.6136 | COSM3405922 |
| | Tyr636Ter | | | |
| ASXL1 | NP_056153.2:p. | NM_015338.5:c.1934dup | 0.2655 | COSM1411076 |
| | Gly646TrpfsTer12 | | | |
| ASXL1 | NP_056153.2:p. | NM_015338.5:c.1934dup | 0.329 | COSM1411076 |
| | Gly646TrpfsTer12 | | | |
| TET2 | NP_001120680.1:p. | NM_001127208.2:c.5152G > T | 0.4937 | COSM41742 |
| | Val1718Leu | | | |
| TET2 | NP_001120680.1:p. | NM_001127208.2:c.5152G > T | 0.529 | COSM41742 |
| | Val1718Leu | | | |
| DDX41 | NP_001308661.1:p. | NM_001321732.1:c.H96G > A | 0.078 | COSM166515 |
| | Arg399His | | | |
| NRAS | NP_002515.1:p. | NM_002524.4:c.35G > A | 0.1757 | COSM564 |
| | Gly12Asp | | | |
| DDX41 | NP_001308661.1:p. | NM_001321732.1:c.1196G > A | 0.0706 | COSM166515 |
| | Arg399His | | | |
| NRAS | NP_002515.1:p. | NM_002524.4:c.35G > A | 0.3419 | COSM564 |
| | Gly12Asp | | | |
| ASXL1 | NP_056153.2:p. | NM_015338.5:c.1900_1922del | 0.4076 | COSM51200 |
| | Glu635ArgfsTer15 | | | |
| CALR | NP_004334.1:p. | NM_004343.3:c.1100_1145del | 0.4974 | COSM1738150 |
| | Leu367GlnfsTer48 | | | |
| ASXL1 | NP_056153.2:p. | NM_015338.5:c.1900_1922del | 0.3958 | COSM51200 |
| | Glu635ArgfsTer15 | | | |
| CALR | NP_004334.1:p. | NM_004343.3:c.1100_1145del | 0.4839 | COSM1738150 |
| | Leu367GlnfsTer48 | | | |
| DNMT3A | NP_001307822.1:p. | NM_001320893.1:c.1285T > A | 0.4745 | COSM231558 |
| | Trp429Arg | | | |
| TET2 | NP_001120680.1:p. | NM_001127208.2:c.3893G > A | 0.501 | COSM87138 |
| | Cys1298Tyr | | | |
| FLT3 | NP_004110.2:p. | NM_004119.2:c.1669G > A | 0.5031 | COSM28043 |
| | Val557Ile | | | |
| TP53 | NP_000537.3:p. | NM_000546.5:c.936del | 0.3396 | COSM2149481 |
| | Ser313AlafsTer32 | | | |
| FLT3 | NP_004110.2:p. | NM_004119.2:c.1669G > A | 0.5106 | COSM28043 |
| | Val557Ile | | | |
| TP53 | NP_000537.3:p. | NM_000546.5:c.936del | 0.287 | COSM2149481 |
| | Ser313AlafsTer32 | | | |
| NRAS | NP_002515.1:p. | NM_002524.4:c.35G > A | 0.4324 | COSM564 |
| | Gly12Asp | | | |
| TP53 | NP_000537.3:p. | NM_000546.5:c.770T > G | 0.8026 | COSM44890 |
| | Leu257Arg | | | |
| NRAS | NP_002515.1:p. | NM_002524.4:c.35G > A | 0.441 | COSM564 |
| | Gly12Asp | | | |
| TP53 | NP_000537.3:p. | NM_000546.5:c.770T > G | 0.5525 | COSM44890 |
| | Leu257Arg | | | |
| ASXL1 | NP_056153.2:p. | NM_015338.5:c.1773C > G | 0.4178 | COSM53200 |
| | Tyr591Ter | | | |
| DNMT3A | NP_001307822.1:p. | NM_001320893.1:c.534G > A | 0.4093 | COSM249799 |
| | Trp178Ter | | | |
| U2AF1 | NP_001020374.1:p. | NM_001025203.1:c.470A > C | 0.3995 | COSM1318797 |
| | Gln157Pro | | | |
| ASXL1 | NP_056153.2:p. | NM_015338.5:c.1773C > G | 0.2361 | COSM53200 |
| | Tyr591Ter | | | |
| DNMT3A | NP_001307822.1:p. | NM_001320893.1:c.534G > A | 0.1903 | COSM249799 |
| | Trp178Ter | | | |
| U2AF1 | NP_001020374.1:p. | NM_001025203.1:c.470A > C | 0.1859 | COSM1318797 |
| | Gln157Pro | | | |
| CDKN2A | NP_478102.2:p. | NM_058195.3:c.316G > A | 0.5302 | COSM12488 |
| | Gly106Arg | | | |
| NRAS | NP_002515.1:p. | NM_002524.4:c.34G > A | 0.0302 | COSM563 |
| | 2Gly1Ser | | | |
| NRAS | NP_002515.1:p. | NM_002524.4:c.34 G > T | 0.3932 | COSM562 |
| | Gly12Cys | | | |
| SRSF2 | NP_001182356.1:p. | NM_001195427.1:c.284C > G | 0.5279 | COSM211661 |
| | Pro95Arg | | | |
| CDKN2A | NP_478102.2:p. | NM_058195.3:c.316G > A | 0.4876 | COSM12488 |
| | Gly106Arg | | | |
| NRAS | NP_002515.1:p. | NM_002524.4:c.34G > A | 0.0492 | COSM563 |
| | Gly12Ser | | | |
| NRAS | NP_002515.1:p. | NM_002524.4:c.34G > T | 0.2769 | COSM562 |
| | Gly12Cys | | | |
| SRSF2 | NP_001182356.1:p. | NM_001195427.1:c.284C > G | 0.4502 | COSM211661 |
| | Pro95Arg | | | |
| NRAS | NP_002515.1:p. | NM_002524.4:c.35G > T | 0.409 | COSM566 |
| | Gly12Val | | | |
| SRSF2 | NP_001182356.1:p. | NM_001195427.1:c.284C > A | 0.4824 | COSM211504 |
| | Pro95His | | | |
| NRAS | NP_002515.1:p. | NM_002524.4:c.35G > T | 0.32 | COSM566 |
| | Gly12Val | | | |
| SRSF2 | NP_001182356.1:p. | NM_001195427.1:c.284C > A | 0.3945 | COSM211504 |
| | Pro95His | | | |
| TP53 | NP_000537.3:p. | NM_000546.5:c.955A > T | 0.5837 | COSM11658 |
| | Lys319Ter | | | |
| ASXL1 | NP_056153.2:p. | NM_015338.5:c.1888_1910del | 0.2704 | COSM51200 |
| | Glu635ArgfsTer15 | CACCACTGCCATAGAGAGGCGGC | | |
| FLT3 | NP_004110.2:p. | NM_004119.2:c.2505T > G | 0.2665 | COSM788 |
| | Asp835Glu | | | |
| FLT3 | NP_004110.2:p. | NM_004119.2:c.970G > A | 0.184 | COSM28040 |
| | Asp324Asn | | | |
| FLT3 | NP_004110.2:p. | NM_004119.2:c.2503G > T | 0.4812 | COSM783 |
| | Asp835Tyr | | | |
| SF3B1 | NP_036565.2:p. | NM_012433.2:c.1998G > T | 0.5254 | COSM131557 |
| | Lys666Asn | | | |
| FLT3 | NP_004110.2:p. | NM_004119.2:c.2503G > T | 0.4795 | COSM783 |
| | Asp835Tyr | | | |
| SF3B1 | NP_036565.2:p. | NM_012433.2:c.1998G > T | 0.5829 | COSM131557 |
| | Lys666Asn | | | |
| RUNX1 | NP_001001890.1:p. | NM_001001890.2:c.511G > A | 0.4668 | COSM24721 |
| | Asp171Asn | | | |
| SRSF2 | NP_001182356.1:p. | NM_001195427.1:c.284C > G | 0.5 | COSM211661 |
| | Pro95Arg | | | |
| RUNX1 | NP_001001890.1:p. | NM_001001890.2:c.511G > A | 0.4769 | COSM24721 |
| | Asp171Asn | | | |
| SRSF2 | NP_001182356.1:p. | NM_001195427.1:c.284C > G | 0.5242 | COSM211661 |
| | Pro95Arg | | | |
| TP53 | NP_000537.3:p. | NM_000546.5:c.773A > C | 0.7886 | COSM1268362 |
| | Glu258Ala | | | |
| GATA2 | NP_001139133.1:p. | NM_001145661.1:c.962T > C | 0.2829 | COSM249844 |
| | Leu321Pro | | | |
| PHF6 | NP_001015877.1:p. | NM_001015877.1:c.955C > T | 0.5612 | COSM144626 |
| | Arg319Ter | | | |
| GATA2 | NP_001139133.1:p. | NM_001145661.1:c.962T > C | 0.2774 | COSM249844 |
| | Leu321Pro | | | |
| PHF6 | NP_001015877.1:p. | NM_001015877.1:c.955C > T | 0.5706 | COSM144626 |
| | Arg319Ter | | | |
| TP53 | NP_000537.3:p. | NM_000546.5:c.817C > T | 0.9103 | COSM99933 |
| | 7Arg23Cys | | | |
| TP53 | NP_000537.3:p. | NM_000546.5:c.817C > T | 0.9476 | COSM99933 |
| | Arg273Cys | | | |
| KRAS | NP_004976.2:p. | NM_004985.3:c.35G > A | 0.1672 | COSM521 |
| | Gly12Asp | | | |
| TP53 | NP_000537.3:p. | NM_000546.5:c.817C > T | 0.267 | COSM99933 |
| | Arg273Cys | | | |
| KRAS | NP_004976.2:p. | NM_004985.3:c.35G > A | 0.1143 | COSM521 |
| | Gly12Asp | | | |
| TP53 | NP_000537.3:p. | NM_000546.5:c.817C > T | 0.1391 | COSM99933 |
| | 7Arg23Cys | | | |
| ASXL1 | NP_056153.2:p. | NM_015338.5:c.3306G > T | 0.4802 | COSM36205 |
| | Glu1102Asp | | | |
| IDH2 | NP_001276839.1:p. | NM_001289910.1:c.263G > A | 0.3356 | COSM41590 |
| | Arg88Gln | | | |
| SRSF2 | NP_001182356.1:p. | NM_001195427.1:c.284C > A | 0.3163 | COSM211504 |
| | Pro95His | | | |
| ASXL1 | NP_056153.2:p. | NM_015338.5:c.3306G > T | 0.499 | COSM36205 |
| | Glu1102Asp | | | |
| IDH2 | NP_001276839.1:p. | NM_001289910.1:c.263G > A | 0.4472 | COSM41590 |
| | Arg88Gln | | | |
| SRSF2 | NP_001182356.1:p. | NM_001195427.1:c.284C > A | 0.4186 | COSM211504 |
| | Pro95His | | | |
| TET2 | NP_001120680.1:p. | NM_001127208.2:c.4393C > T | 0.2072 | COSM42016 |
| | Arg1465Ter | | | |
| TET2 | NP_001120680.1:p. | NM_001127208.2:c.4393C > T | 0.2399 | COSM42016 |
| | Arg1465Ter | | | |
| ASXL1 | NP_056153.2:p. | NM_015338.5:c.1934dup | 0.387 | COSM1411076 |
| | Gly646TrpfsTer12 | | | |
| NRAS | NP_002515.1:p. | NM_002524.4:c.35G > A | 0.5068 | COSM564 |
| | Gly12Asp | | | |
| SRSF2 | NP_001182356.1:p. | NM_001195427.1:c.284C > A | 0.5192 | COSM211504 |
| | Pro95His | | | |
| ASXL1 | NP_056153.2:p. | NM_015338.5:c.1934dup | 0.1854 | COSM1411076 |
| | Gly646TrpfsTer12 | | | |
| NRAS | NP_002515.1:p. | NM_002524.4:c.35G > A | 0.1212 | COSM564 |
| | Gly12Asp | | | |
| SRSF2 | NP_001182356.1:p. | NM_001195427.1:c.284C > A | 0.2282 | COSM211504 |
| | Pro95His | | | |
| CDKN2A | NP_000068.1:p. | NM_000077.4:c.9_32dup | 0.259 | COSM13442 |
| | Ala4Pro11dup | | | |
| DNMT3A | NP_001307822.1:p. | NM_001320893.1:c.2188C > T | 0.2769 | COSM53042 |
| | Arg730Cys | | | |
| TP53 | NP_000537.3:p. | NM_000546.5:c.395A > G | 0.0644 | COSM1646844 |
| | Lys132Arg | | | |
| TET2 | NP_001120680.1:p. | NM_001127208.2:c.5152G > T | 0.0903 | COSM41742 |
| | Val1718Leu | | | |
| TP53 | NP_000537.3:p. | NM_000546.5:c.524G > A | 0.6381 | COSM99022 |
| | Arg175His | | | |
| TET2 | NP_001120680.1:p. | NM_001127208.2:c.5152G > T | 0.3953 | COSM41742 |
| | Val1718Leu | | | |
| TP53 | NP_000537.3:p. | NM_000546.5:c.524G > A | 0.0766 | COSM99022 |
| | Arg175His | | | |
| TP53 | NP_000537.3:p. | NM_000546.5:c.743G > A | 0.6071 | COSM99602 |
| | Arg248Gln | | | |
| TP53 | NP_000537.3:p. | NM_000546.5:c.743G > A | 0.8388 | COSM99602 |
| | Arg248Gln | | | |
| IDH2 | NP_001276839.1:p. | NM_001289910.1:c.263G > A | 0.3684 | COSM41590 |
| | Arg88Gln | | | |
| JAK2 | NP_001309123.1:p. | NM_001322194.1:c.3323A > G | 0.3958 | COSM33708 |
| | Asn1108Ser | | | |
| NPM1 | NP_002511.1:p. | NM_002520.6:c.860_863dup | 0.2594 | COSM158604 |
| | Trp288CysfsTer12 | | | |
| ASXL1 | NP_056153.2:p. | NM_015338.5:c.1934dup | 0.3645 | COSM1411076 |
| | Gly646TrpfsTer12 | | | |
| DNMT3A | NP_001307822.1:p. | NM_001320893.1:c.1447C > T | 0.4696 | COSM1407108 |
| | Arg483Trp | | | |
| RUNX1 | NP_001001890.1:p. | NM_001001890.2:c.238C > T | 0.4816 | COSM24736 |
| | Arg80Cys | | | |
| TET2 | NP_001120680.1:p. | NM_001127208.2:c.5059C > T | 0.4837 | COSM43421 |
| | Gln1687Ter | | | |
| NRAS | NP_002515.1:p. | NM_002524.4:c.35G > A | 0.0742 | COSM564 |
| | Gly12Asp | | | |
| DNMT3A | NP_001307822.1:p. | NM_001320893.1:c.2189G > A | 0.4209 | COSM52944 |
| | Arg730His | | | |
| NPM1 | NP_002511.1:p. | NM_002520.6:c.860_863dup | 0.2382 | COSM158604 |
| | Trp288CysfsTer12 | | | |
| SF3B1 | NP_036565.2:p. | NM_012433.2:c.1997A > G | 0.4156 | COSM131553 |
| | Lys666Arg | | | |
| TP53 | NP_000537.3:p. | NM_000546.5:c.916C > T | 0.5947 | COSM3388168 |
| | Arg306Ter | | | |
| TP53 | NP_000537.3:p. | NM_000546.5:c.916C > T | 0.6155 | COSM3388168 |
| | Arg306Ter | | | |
| TP53 | NP_000537.3:p. | NM_000546.5:c.488A > G | 0.4817 | COSM10808 |
| | Tyr163Cys | | | |
| TP53 | NP_000537.3:p. | NM_000546.5:c.488A > G | 0.5308 | COSM10808 |
| | Tyr163Cys | | | |
| KRAS | NP_004976.2:p. | NM_004985.3:c.179G > T | 0.0422 | COSM1667041 |
| | Gly60Val | | | |
| CSF3R | NP_000751.1:p. | NM_000760.3:c.1853C > T | 0.3521 | |
| | Thr618Ile | | | |
| ASXL1 | NP_056153.2:p. | NM_015338.5:c.1934dup | 0.3722 | COSM1411076 |
| | Gly646TrpfsTer12 | | | |
| DNMT3A | NP_001307822.1:p. | NM_001320893.1:c.1447C > T | 0.4212 | COSM1407108 |
| | Arg483Trp | | | |
| RUNX1 | NP_001001890.1:p. | NM_001001890.2:c.238C > T | 0.4395 | COSM24736 |
| | Arg80Cys | | | |
| TET2 | NP_001120680.1:p. | NM_001127208.2:c.5059C > T | 0.4674 | COSM43421 |
| | Gln1687Ter | | | |
| ASXL1 | NP_056153.2:p. | NM_015338.5:c.1934dup | 0.3434 | COSM1411076 |
| | Gly646TrpfsTer12 | | | |
| DNMT3A | NP_001307822.1:p. | NM_001320893.1:c.1447C > T | 0.483 | COSM1407108 |
| | Arg483Trp | | | |
| RUNX1 | NP_001001890.1:p. | NM_001001890.2:c.238C > T | 0.4739 | COSM24736 |
| | Arg80Cys | | | |
| TET2 | NP_001120680.1:p. | NM_001127208.2:c.5059C > T | 0.4643 | COSM43421 |
| | Gln1687Ter | | | |
| SF3B1 | NP_036565.2:p. | NM_012433.2:c.2098A > G | 0.0272 | COSM84677 |
| | Lys700Glu | | | |
| SF3B1 | NP_036565.2:p. | NM_012433.2:c.1998G > C | 0.3802 | COSM132937 |
| | Lys666Asn | | | |
| TET2 | NP_001120680.1:p. | NM_001127208.2:c.4076G > A | 0.0903 | COSM42055 |
| | Arg1359His | | | |
| U2AF1 | NP_001020374.1:p. | NM_001025203.1:c.467G > A | 0.0831 | COSM1235014 |
| | Arg156His | | | |
| SF3B1 | NP_036565.2:p. | NM_012433.2:c.1998G > C | 0.4081 | COSM132937 |
| | Lys666Asn | | | |
| TET2 | NP_001120680.1:p. | NM_001127208.2:c.4076G > A | 0.0671 | COSM42055 |
| | Arg1359His | | | |
| U2AF1 | NP_001020374.1:p. | NM_001025203.1:c.467G > A | 0.1158 | COSM1235014 |
| | Arg156His | | | |
| CBL | NP_005179.2:p. | NM_005188.3:c.1253T > C | 0.1436 | COSM34070 |
| | Phe418Ser | | | |
| NPM1 | NP_002511.1:p. | NM_002520.6:c.860_863dup | 0.1184 | COSM158604 |
| | Trp288CysfsTer12 | | | |
| STAG2 | | NM_001042749.1:c.386-8A > G | 0.2622 | COSM1717794 |
| ASXL1 | NP_056153.2:p. | NM_015338.5:c.1934dup | 0.3516 | COSM1411076 |
| | Gly646TrpfsTer12 | | | |
| ASXL1 | NP_056153.2:p. | NM_015338.5:c.4099G > A | 0.4931 | COSM723114 |
| | Val1367Ile | | | |
| NRAS | NP_002515.1:p. | NM_002524.4:c.34G > C | 0.4048 | COSM561 |
| | Gly12Arg | | | |
| SETBP1 | NP_056374.2:p. | NM_015559.2:c.2608G > A | 0.4365 | COSM1234973 |
| | Gly870Ser | | | |
| SRSF2 | NP_001182356.1:p. | NM_001195427.1:c.284C > T | 0.5906 | COSM211028 |
| | Pro95Leu | | | |
| ASXL1 | NP_056153.2:p. | NM_015338.5:c.1934dup | 0.3822 | COSM1411076 |
| | Gly646TrpfsTer12 | | | |
| ASXL1 | NP_056153.2:p. | NM_015338.5:c.4099G > A | 0.5358 | COSM723114 |
| | Val1367Ile | | | |
| NRAS | NP_002515.1:p. | NM_002524.4:c.34G > C | 0.367 | COSM561 |
| | Gly12Arg | | | |
| SETBP1 | NP_056374.2:p. | NM_015559.2:c.2608G > A | 0.47 | COSM1234973 |
| | Gly870Ser | | | |
| SRSF2 | NP_001182356.1:p. | NM_001195427.1:c.284C > T | 0.5972 | COSM211028 |
| | Pro95Leu | | | |
| DNMT3A | NP_001307822.1:p. | NM_001320893.1:c.2189G > A | 0.4626 | COSM52944 |
| | Arg730His | | | |
| IDH2 | NP_001276839.1:p. | NM_001289910.1:c.359G > A | 0.4742 | COSM33733 |
| | Arg120Lys | | | |
| DNMT3A | NP_001307822.1:p. | NM_001320893.1:c.2189G > A | 0.5147 | COSM52944 |
| | Arg730His | | | |
| IDH2 | NP_001276839.1:p. | NM_001289910.1:c.359G > A | 0.481 | COSM33733 |
| | Arg120Lys | | | |
| RUNX1 | NP_001001890.1:p. | NM_001001890.2:c.877C > T | 0.3757 | COSM41699 |
| | Arg293Ter | | | |
| RUNX1 | NP_001001890.1:p. | NM_001001890.2:c.877C > T | 0.3237 | COSM41699 |
| | Arg293Ter | | | |

TABLE 13
Mutational profile of patients achieving CR or CRi (n = 6)

| Patient ID | Mutations |
|---|---|
| 03-037 | DNMT3A, TET2, NPM1 |
| 05-030 | SRSF2, NRAS, CDKN2A |
| 07-009 | SF3B1, FLT3 |
| 07-035 | ASXL1, SRSF2, IDH2 |
| 08-058 | NRAS |
| 09-064 | SF3B1 |

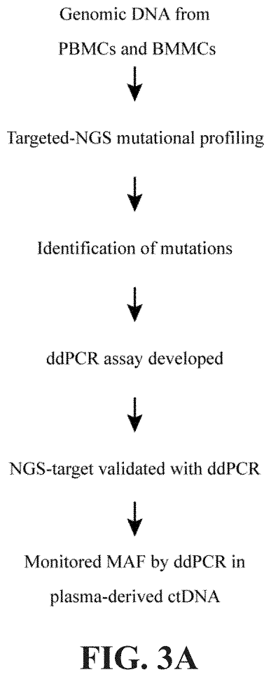

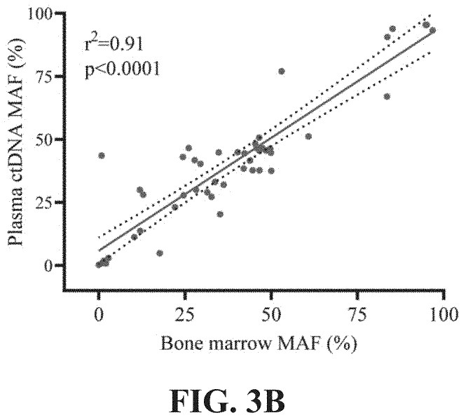

Recent studies have shown that plasma-derived ctDNA reflects the fractional abundance of somatic mutations detected in bone marrow in AML and MDS. For 20 patients enrolled in this study, a driver mutation was identified by targeted NGS using gDNA from bone marrow or blood, and the MAF of the selected variant was measured in ctDNA from plasma by ddPCR (FIG. 3A). In agreement with prior studies, a strong correlation (r2=0.91; P<0.0001) was observed between the MAF measured by ddPCR in bone marrow and ctDNA across 49 matched samples (FIG. 3B), supporting the use of ctDNA as a noninvasive tool to monitor treatment response.

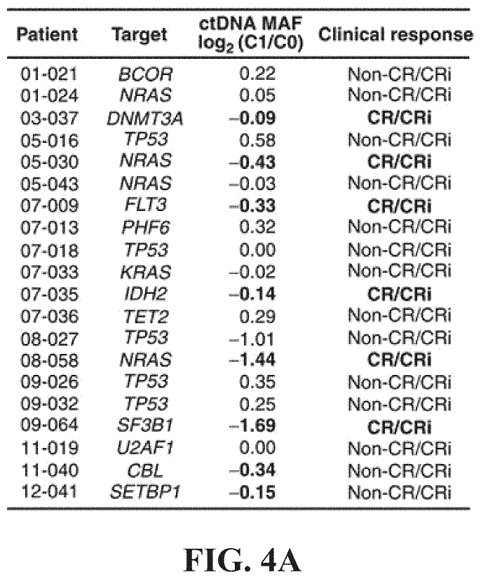

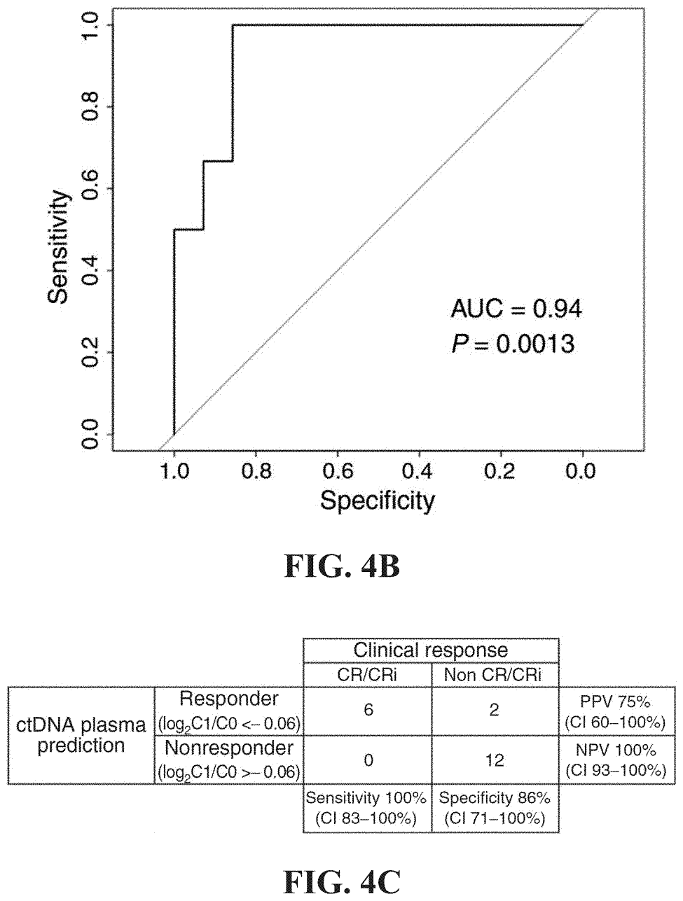

Utility of measuring early changes in ctDNA MAF to predict clinical response (CR/CRi) was determined. For this purpose, MAF was assessed in ctDNA before beginning treatment (C0) and after 1 cycle of treatment (C1), and change in MAF calculated as log₂(C₁/C₀; FIG. 4A). Using an ROC analysis, we showed that changes in ctDNA MAF at cycle 1 were predictive of clinical responses (AUC=0.94; P=0.0013; FIG. 4B). The optimal log₂(C₁/C₀) threshold was calculated to be −0.06, at which the change in MAF predicted patient clinical response with 86% specificity (71%-100% C1) and 100% sensitivity (83%-100% CI; FIG. 4C). Positive and negative predictive values were 75% (60%-100% CI) and 100% (93%-100% CI), respectively. All patients with clinical response (n=6) showed a decrease in ctDNA MAF at cycle 1 [log₂(C₁/C₀)<−0.06], while only two of the 14 nonresponders showed a similar decrease (FIGS. 4A-C). The median number of cycles to achieve CR/CRi was 3 (range, 1-7) with only 1 patient achieving CR after 1 cycle. Altogether, these data suggest that changes in ctDNA MAF after the first cycle are associated with later clinical response and have the potential to help guide patient treatment.

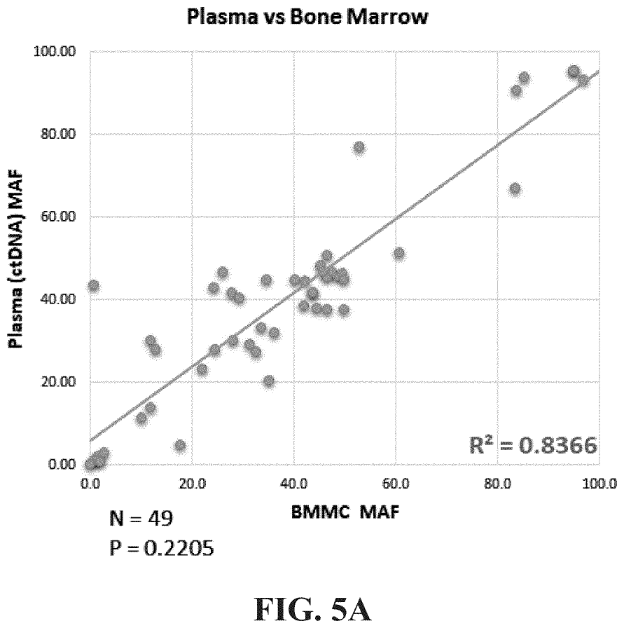

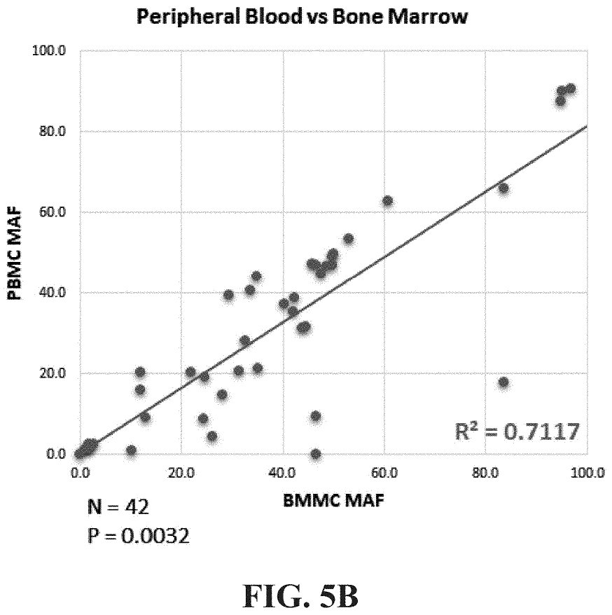

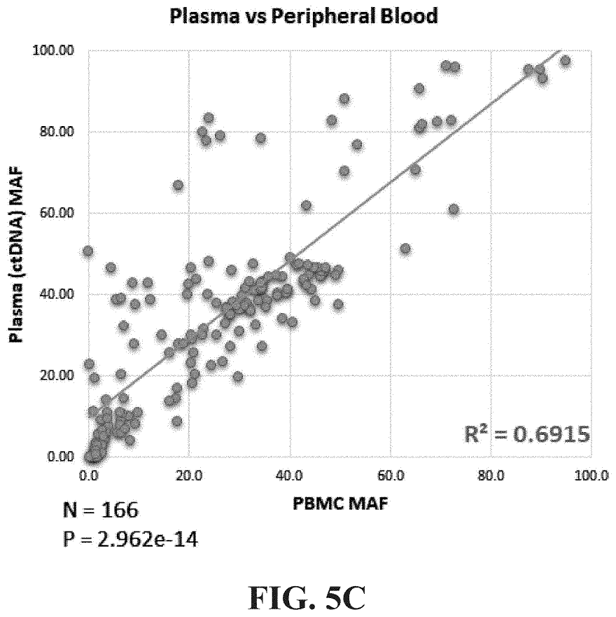

It was found that plasma-derived ctDNA mutation allelic frequencies (MAF) highly correlates with MAF from bone marrow mononuclear cells. MAF in ctDNA from plasma, and gDNA from BMMCs and PMBCS were assessed for 20 patients across timepoints (Plasma, BM, PB). Linear regression, and a paired two sample t-test were performed for all timepoints with matched samples. When plotted (FIGS. 5A-C), MAF from plasma and BM showed the highest linearity (R²=0.8366) and no significant difference from BM (P=0.2205) when comparing 49 matched samples. This data indicate that plasma is representative of disease state in BM, and can be used to monitor response to treatment.

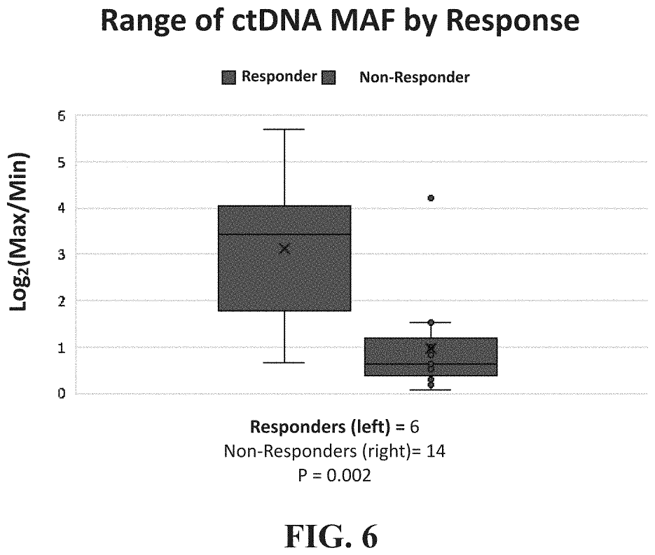

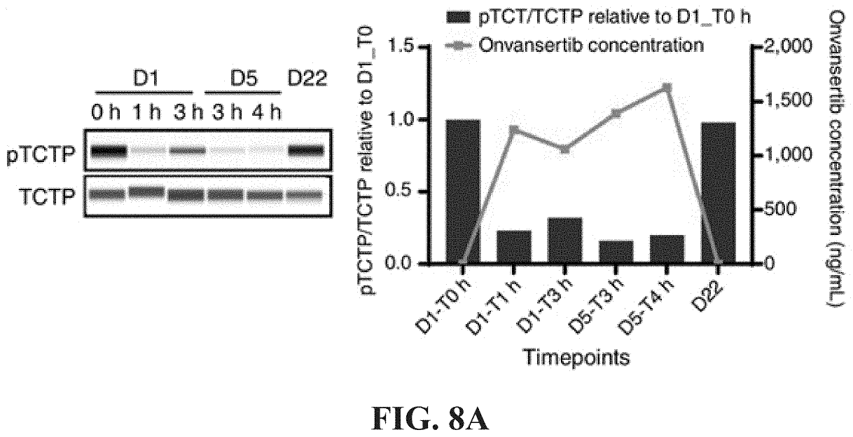

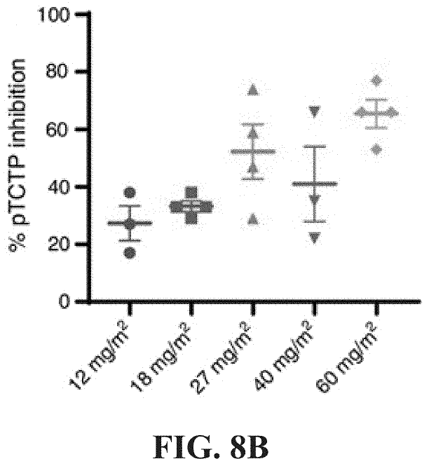

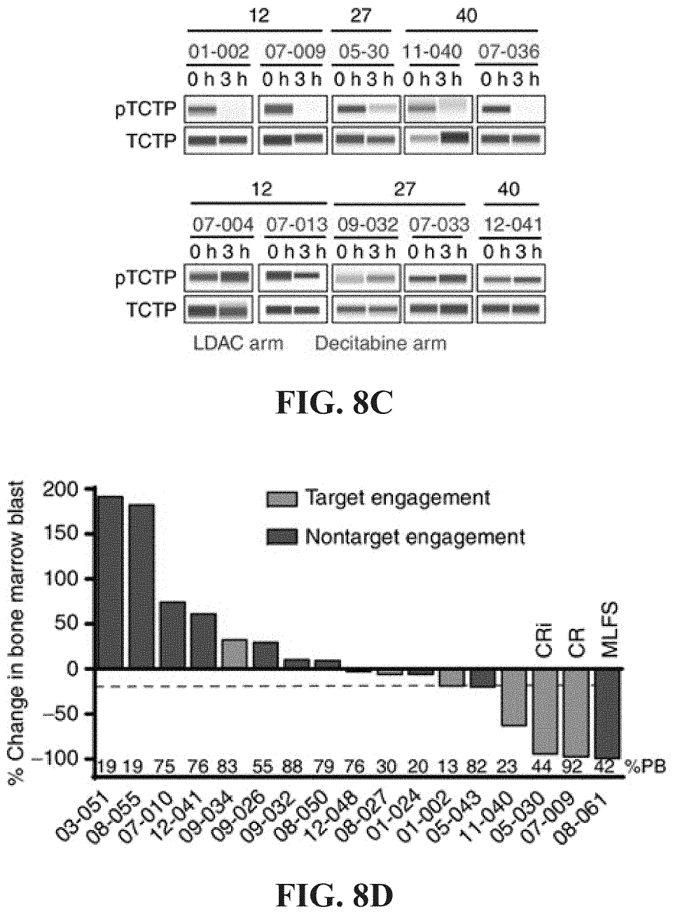

It was also found that responders have a greater range of ctDNA MAF than Non-responders. After plasma was found to be correlative with BM, it was further determined that changes in plasma MAF differed between patients with clinical response (CR/CRi) and non-responders. As shown in FIG. 6, the change in MAF over all timepoints available for each patients was log transformed: Log₂(Max/Min), where Max is highest MAF of all timepoints, and Min is the lowest MAF of all timepoints. A t-test was performed between responders (n=6) and non-responders (n=14) to determine if ctDNA range distinguished the groups (FIGS. 8A-D). As shown in FIGS. 8A-D, there was a significant difference between the two groups (P=0.002), further supporting ctDNA as a biomarker for treatment response.

It was believed that plasma-derived ctDNA is a predicative biomarker for treatment response to ovansertib, and the utility of plasma MAF to predict clinical response after the first cycle of treatment was determined. MAF were assessed in ctDNA before beginning treatment (C0) and after 1 cycle of treatment (C1) when BM aspirates were collected; and change in MAF calculated as Log₂(C₁/C₀). Similarly changes in % BM blasts at end of cycle 1 versus screening were calculated as Log₂(C₁/C₀). The results are shown below in Table 14.

TABLE 14
Clinical outcome of patients

| | ctDNA MAF | BM % Blasts | |
|---|---|---|---|
| Patient | Log₂(C₁/C₀) | Log₂(C₁/C₀) | Clinical Response |
| 01-021 | 0.2193 | −0.8625 | NR |
| 01-024 | 0.0489 | −0.3219 | NR |
| 03-037 | −0.0853 | 0.8301 | Responder |
| 05-016 | 0.5850 | 3.0704 | NR |
| 05-030 | −0.4258 | −2.0000 | Responder |
| 05-043 | −0.0299 | −0.4150 | NR |
| 07-009 | −0.3303 | −0.0468 | Responder |
| 07-013 | 0.3182 | N/A | NR |
| 07-018 | 0.0015 | N/A | NR |
| 07-033 | −0.0200 | N/A | NR |
| 07-035 | −0.1410 | −1.7370 | Responder |
| 07-036 | 0.2940 | −1.0000 | NR |
| 08-027 | −0.0096 | −0.1043 | NR |
| 08-058 | −1.4399 | 0.9329 | Responder |
| 09-026 | 0.3531 | 0.3785 | NR |
| 09-032 | 0.2534 | 0.1454 | NR |
| 09-064 | −1.6881 | −3.9069 | Responder |
| 11-019 | 0.0000 | 0.0000 | NR |
| 11-040 | −0.3411 | 0.4021 | NR |
| 12-041 | −0.1515 | 0.6951 | NR |
| | N = 20 | N = 17 | NR = Non-responder |

Results of screening test using ctDNA MAF after 1 cycle are shown in Table 15, which shows that all patients with clinical response (CR+CRi, n=6) had a decrease in ctDNA MAF at cycle 1 (Log₂(C₁/C₀)<−0.05), while only 2 of the 14 non-responders (NR) showed a similar decrease. Measuring the changes in plasma over the first cycle predicted patient response with 90% diagnostic accuracy, 100% sensitivity and 86% specificity, with positive predictive value (75%) and negative predictive value (100%) supporting the utility of this analysis. Screening test were conducted using % BM blasts after 1 cycle, and the results are shown in are shown in Table 16 and indicate that the same analysis in BM Blasts at Cycle 1 were not predictive of clinical response. For the analysis of test results, the following are used:

$$Sensitivity = \frac{TP}{( TP + FN )}$$

$$Specificity = \frac{TN}{( TN + FP )}$$

$$Positive Predictive Value ( PPV ) = \frac{TP}{( TP + FP )}$$

$$Negative Predictive Value ( NPV ) = \frac{TN}{( TP + TN )}$$

$$Diagnostic Accuracy ( DA ) = \frac{TP + TN}{TP + TN + FP + FN}$$

$$Positive Likelihood Ratio ( PLR ) = \frac{Sensitivity}{( 1 - Specificity )}$$

$$Negative Likelihood Ration ( NLR ) = \frac{( 1 - Sensitivity )}{Specificity}$$

TABLE 15
Screening test using ctDNA MAF after 1 cycle.

| | | Clinical Response | | | |
|---|---|---|---|---|---|
| | | Responder | NR | | |
| Plasma Prediction | Responder | 6 | 2 | PPV | 75% |
| | NR | 0 | 12 | NPV | 100% |
| | | 100% | 86% | | |
| | | Sensitivity | Specificity | | |

| | |
|---|---|
| 90% | Diagnostic accuracy |
| 7.0 | Positive likelihood ratio |
| 0.0 | Negative likelihood ratio |

TABLE 16
Screening test using % BM blasts after 1 cycle.

| | | Clinical Response | | | |
|---|---|---|---|---|---|
| | | Responder | NR | | |
| BM Prediction | Responder | 3 | 5 | PPV | 38% |
| | NR | 3 | 6 | NPV | 67% |
| | | 50% | 55% | | |
| | | Sensitivity | Specificity | | |

| | |
|---|---|
| 53% | Diagnostic accuracy |
| 1.10 | Positive likelihood ratio |
| 0.9 | Negative likelihood ratio |

Earlier Treatment Decisions from Serial Monitoring of ctDNA

As shown herein, plasma-derived ctDNA enables monitoring of treatment response and disease progression.

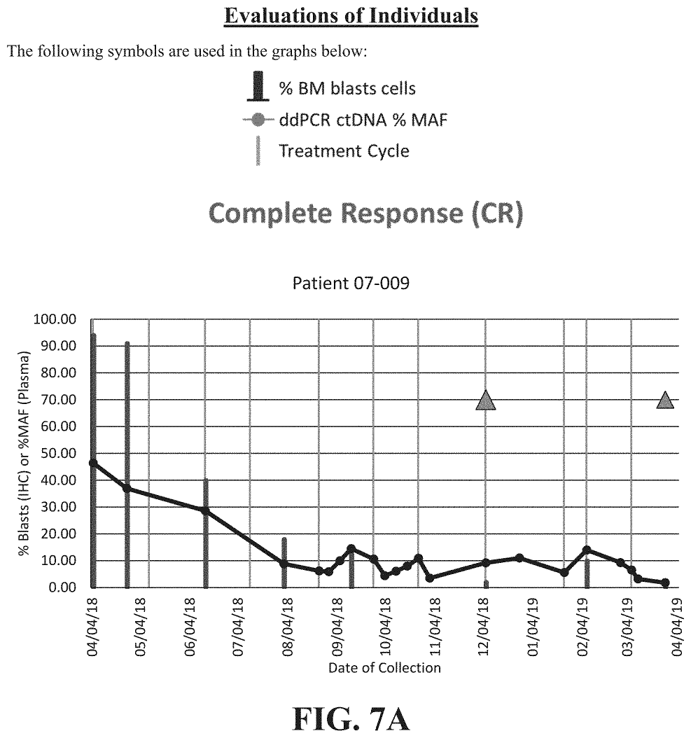

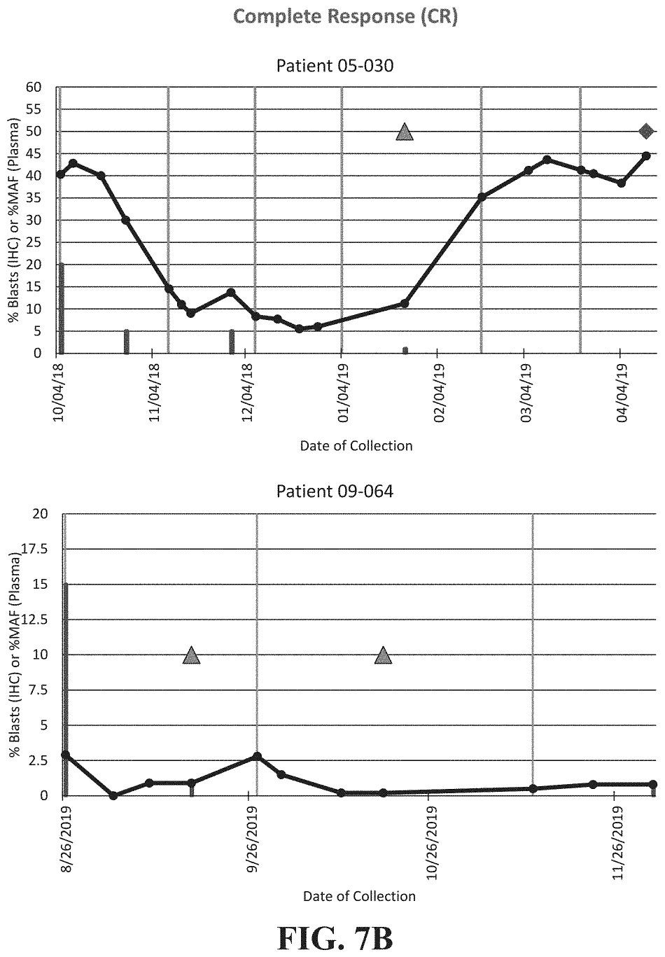

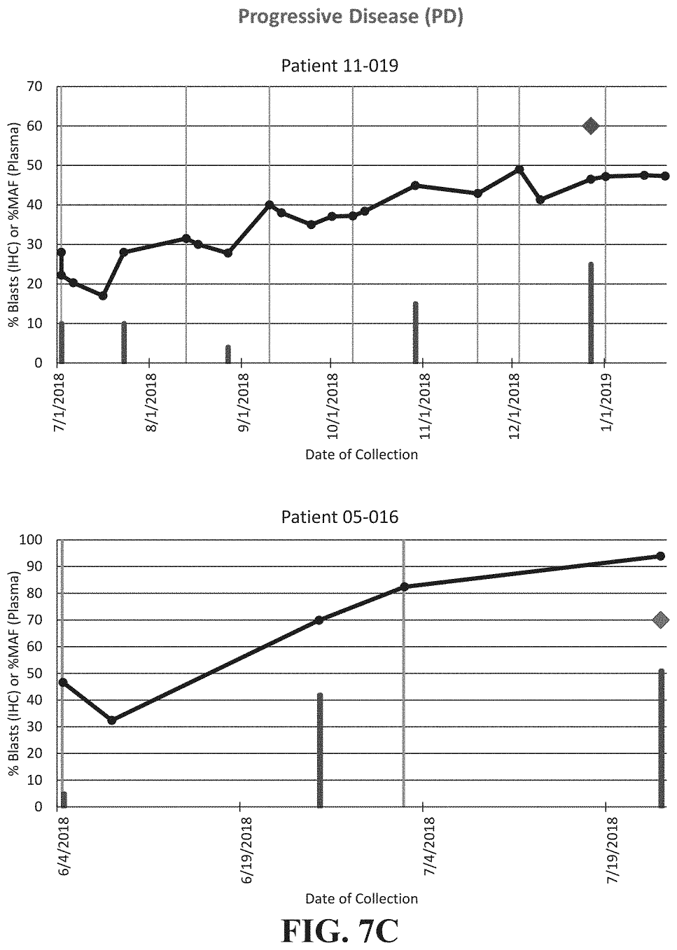

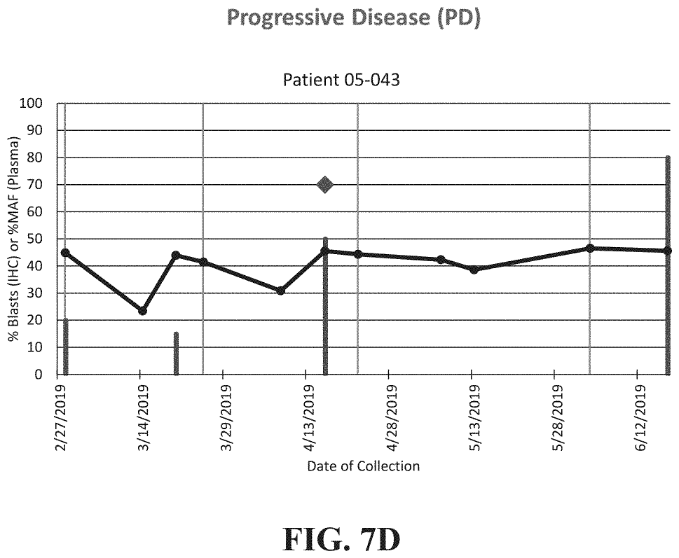

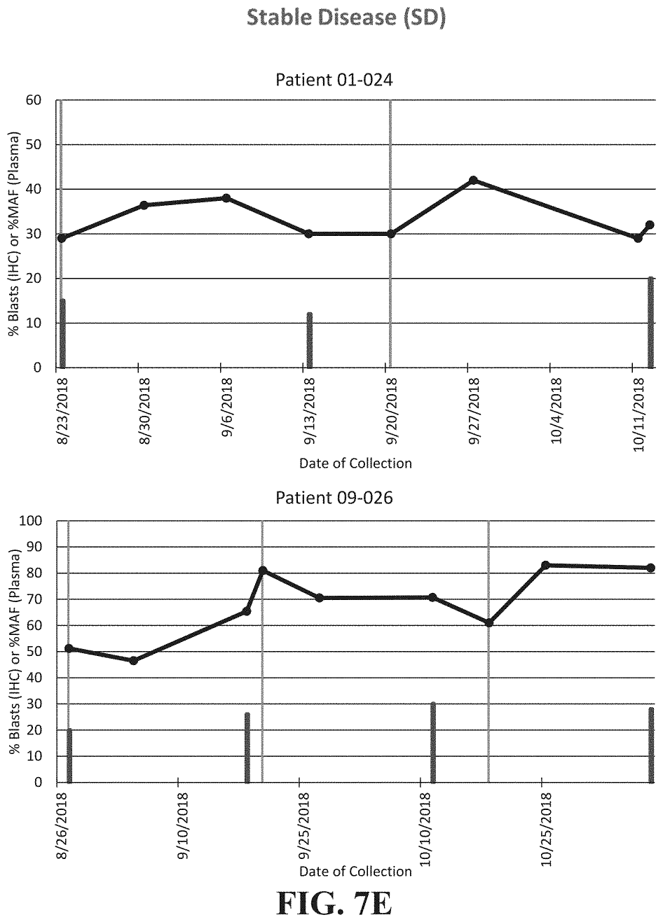

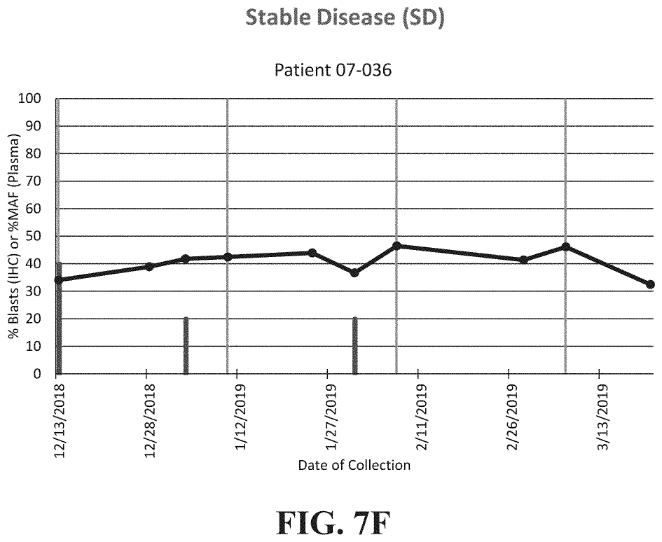

Twenty patients were chosen for ctDNA monitoring (see Table 17). For each patient, plots were generated overlaying ctDNA MAF and % BM blasts by date of sample collection. Results are shown in FIGS. 7A-F. Of 6 patients who reached CR/CRi (triangles in FIGS. 7A-B), all showed the lowest ctDNA MAF at or before (13-35 days) determination of clinical response. Similarly, 4 patients with Progressive disease (PD, diamonds in FIGS. 7C-D), displayed a maximum ctDNA MAF at or before determination of progression.

TABLE 17
ctDNA analysis results

| Patient | ctDNA MAF Min (Days before CR/CRi) |
|---|---|
| 07-009 | 35 |
| 05-030* | 34 |
| 03-037 | 21 |
| 09-064 | 13 |
| 07-035 | 0 |
| 08-058 | 0 |
| Patient | ctDNA MAF Max (Days before PD) |
| 11-019 | 24 |
| 05-043 | 14 |
| 05-030* | 0 |
| 05-016 | 0 |

As shown in Table 17, for Patient 05-030, minimum ctDNA MAF was reached 34 days prior to CR diagnosis. A spike in ctDNA MAF was detected 53 days before progression, with a maximum MAF at the time of progression.

Patients with stable disease also show ctDNA trends resembling the bone marrow. Patients who responded to treatment showed a ctDNA MAF trend that decreased, while those who progressed showed an increasing trend. Therefore, ctDNA MAF is capable of monitoring both clinical response and disease progression, and often prior to the bone marrow biopsy.

Target Engagement is Associated with Greater Decrease in Bone Marrow Blasts

The plasma inhibitory activity assay was used to determine whether a sufficient amount of free drug was present in the plasma of patients to inhibit phosphorylation of the PLK1 substrate, TCTP. In preclinical models, onvansertib inhibits pTCTP in vitro and in vivo. We confirmed that pTCTP was reduced by on-treatment plasma (days 1 and 5), but not by plasma with undetectable onvansertib levels (day 22; FIG. 8A). In addition, pTCTP inhibition was observed using plasma from all dose levels (12-60 mg/m²) with greater pTCTP inhibition at higher doses, correlating with inhibition of PLK1 activity (FIG. 8B).

Target engagement in blasts was determined by measuring relative pTCTP/TCTP protein levels in PBMCs isolated from patient blood prior to and 3 hours after the first dose of onvansertib (FIG. 8C). Target engagement was defined as a decrease of ≥50% in pTCTP/TCTP at 3 hours postdose versus predose in patients with at least 10% of circulating blasts, and was observed in 8 (33%) of the 24 evaluable patients. Interestingly, target engagement was not dependent on onvansertib dose, pharmacokinetics, or combination treatment (FIG. 8C). However, it seemed to be associated with higher decrease in bone marrow blasts: 4 (67%) of the 6 target engagement patients had a ≥20% decrease in blasts versus 2 (18%) of the 11 nontarget engagement patients (FIG. 8D). Although the difference between the two groups was not significant (Fisher exact test, P=0.11), these data suggest a potential link between target engagement and antileukemic activity that should be further explore.

### Conclusion

As demonstrated herein, mutant allele frequency (MAF) in plasma-derived ctDNA was highly correlated with MAF in gDNA from BM cells (R²=0.8366). Patients with clinical response (CR/CRi) showed bigger changes in ctDNA MAF than non-responders, suggesting that ctDNA changes can be used as a surrogate for treatment response.

It was found that change in ctDNA MAF after the 1st cycle of treatment was highly predictive of response to onvansertib. All patients achieving CR/CRi (n=6) had a decrease in ctDNA MAF after 1 cycle (Log₂(C₁/C₀)<−0.05). In addition, 86% of the non-responders (12 out of 14) showed no decrease in ctDNA MAF after 1 cycle.

Serial monitoring in plasma provides a less-invasive alternative to bone marrow, often enabling earlier detection of clinical response or progression, since minimum ctDNA MAF was measured at or before remission, while maximum ctDNA MAF was measured at or before progression.

As shown here, effective treatment options for patients with R/R AML are limited, with generally low CR and limited response durations leading to dismal survival. PLK1 has been identified as a promising target for AML in preclinical studies, however, nonspecific PLK1 inhibitors have shown high toxicity profiles and failed in clinic. The failure of the pan-PLK inhibitor, volasertib, in a phase III trial may have stemmed from its long half-life (˜5 days) and a consequent inability to adequately control the extended myelosuppression, from the absence of response biomarkers to select patients, and from the lack of specificity for PLK1 resulting in inhibition of PLK2 and PLK3, both mediators of DNA damage or oxidative stress that have tumor suppressor functions. Onvansertib with its high specificity for PLK1, its bioavailability, and short half-life, has the potential to mitigate toxicities observed with previous nonspecific PLK1 inhibitors. In this phase Ib clinical trial, patients with R/R AML, or de novo AML considered ineligible for intensive chemotherapy, were treated with onvansertib in combination with either LDAC or decitabine. In this example, safety, pharmacokinetics, and preliminary antileukemic activity of these combinations were evaluated. In addition, novel biomarkers reflecting onvansertib activity were identified and determined as useful in predicting clinical response to therapy.

Patients treated in this trial were characterized predominantly by poor-risk features, including advanced age (median age of 68 years), adverse risk karyotype (68%), and high-risk mutations (28% TP53, 20% ASXL1, 16% FLT3, and 8% RUNX1).

Onvansertib was well tolerated at the first five dose levels (12-60 mg/m²) in combination with both LDAC and decitabine. The most commonly reported G3/G4 AEs were hematologic (neutropenia, thrombocytopenia, and anemia), which are all expected in patients with AML and consistent with the known myelosuppression of decitabine, LDAC, and single-agent onvansertib in patients with solid tumors. Onvansertib-related gastrointestinal disorders (nausea, vomiting, and diarrhea) were generally mild to moderate and manageable, with no patient discontinuing therapy due to these events. Although no skin toxicities were seen in the single-agent phase I study, where the maximum onvansertib dose administered was 48 mg/m²; in this study, skin toxicities were observed at the last two dose levels of onvansertib (60 and 90 mg/m²), with a higher incidence and higher grade at 90 mg/m², suggesting that this should be considered a side-effect of high onvansertib levels.

The onvansertib and decitabine combination showed preliminary antileukemic activity with ORR of 33% across all dose levels (four CR, one CRi, one MLFS, and one PR), while the onvansertib and LDAC combination had less clinical activity (ORR 11%; one CRi and one PR). Historically, the CR/CRi rate of patients with R/R AML to HMAs is 16%, with no significant difference between azacitidine and decitabine. In the decitabine arm of our study, the CR/CRi rate of 24% observed across all doses was encouraging, yet further exploration of the combination is warranted to determine its benefit over HMAs. On the basis of these preliminary efficacy data and the fact that standard of care is currently more aligned with the use of decitabine in the United States, the decitabine combination was selected to be further explored in the phase II and the LDAC arm was discontinued.

Molecular disease monitoring using ctDNA was shown to reflect tumor burden during therapy in MDS and AML. Similarly, we observed a high correlation between MAF in the bone marrow and ctDNA at matched timepoints. In addition, we found that a decrease in MAF of ctDNA after the first cycle of treatment was associated with clinical response, regardless of the time of response. We recognize that this study has several limitations, including the small sample size to perform the ROC analysis and the lack of an independent cohort to validate the threshold obtained with the ROC analysis. The utility of serial ctDNA measurements as a predictor of clinical response should be further explored as it may help guide treatment for patients with AML.

A decrease in pTCTP was shown to parallel PLK1 inhibition by onvansertib in in vitro and in vivo preclinical models. Onvansertib plasma inhibitory activity was correlated with plasma drug levels, albeit not with clinical response. Conversely, target engagement in circulating blasts was observed in a subset of patients and was not associated with onvansertib dose, but with a greater decrease in bone marrow blasts. This observation may be due to the fact that PLK1 activity in leukemic blasts varies between patients resulting in a range of sensitivity and response to PLK1 inhibitors. Further development of the target engagement assay and its evaluation in a larger patient population are needed to determine its true utility as response biomarker.

Recently, venetoclax in combination with HMAs or LDAC has been introduced for older patients ineligible for intensive chemotherapy. However, the median OS of patients after failure of venetoclax in combination with HMAs is low, and novel therapies are urgently needed for this population. Onvansertib has shown efficacy in venetoclax-resistant preclinical models, supporting its potential for venetoclax R/R AML patients. A phase II study of 32 patients treated with onvansertib (60 mg/m²) and decitabine is currently ongoing to further assess the safety profile and antileukemic activity of this combination in patients with R/R AML, including patients who failed prior venetoclax therapy. The identification of predictive biomarkers would be key to enrich for patients who are likely to benefit from the onvansertib and decitabine combination. The ongoing phase II correlative studies include further evaluation of the association between target engagement, changes in ctDNA and clinical response, and identification of additional potential biomarkers through genomic and transcriptional analyses.

### Example 2

### Additional Analyses of Phase 1b/2 Study of Onvansertib, in Combination with LDAC or Decitabine, in Treating AML

Patient enrollment and baseline characteristics: Table 18 shows the number of patients treated in Phase 1b/2 as of Nov. 5, 2020, and patient baseline characteristics are shown in Table 19.

TABLE 18
Patient enrollment

| Phase 1b | Treated patients |
|---|---|
| 12 mg/m² | 4 |
| 18 mg/m² | 3 |
| 27 mg/m² | 3 |
| 40 mg/m² | 4 |
| 60 mg/m² | 3 |
| 90 mg/m² | 6 |
| Phase 2 | Treated patients |
| 60 mg/m² | 31 |

According to the Phase 1b study disclosed herein, MTD was established at 60 mg/m². Treatment was well tolerated through the first 5 dose levels (12-60 mg/m²), and 2 of the 6 patients treated at 90 mg/m² had a DLT (G3 mucositis and G4 rash).

Safety analysis were conducted at the MTD, 60 mg/m² (n=34) and the results are shown in Table 20. As shown in Table 20, most frequent Grade 3-4 AEs were hematological AEs, febrile neutropenia, lung infection and skin toxicities (rash, stomatitis). Two patients discontinued treatment due to Grade 3 rash. 7 deaths occurred in patients while on treatment, all of which were related to AML or its complications. All-causality Treatment-Related Adverse Events Reported in >10% patients (patients treated at the RP2D, n=34).

TABLE 19
Patient baseline characteristics

| N (%) or median [range] | Phase 1b (n = 23) | Phase 2 (n = 31) |
|---|---|---|
| Age, years | 66 [33-77] | 73 [23-85] |
| Male gender | 15 (65%) | 18 (58%) |
| ECOG 0-1 | 22 (96%) | 22 (71%) |
| ECOG 2 | 1 (4%) | 9 (29%) |
| Prior treatment | | |
| 0 | 4 (17%) | 2 (6%) |
| 1 | 13 (57%) | 27 (87%) |
| 2+ | 6 (26%) | 1 (3%) |
| Prior HMA treatment* | 6 (26%) | 15 (48%) |
| Prior Venetoclax treatment** | 0 (0) | 10 (32%) |
| Cytogenetic Risk | | |
| Favorable | 1 (4%) | 3 (10%) |
| Intermediate | 9 (39%) | 7 (23%) |
| Adverse | 13 (57%) | 17 (55) |
| Unknown | 0 (0%) | 4 (13%) |

*for AML or MDS diseases
**for AML disease

TABLE 20
Safe results

| Adverse Events | Grade 1-2 | Grade 3 | Grade 4 | All Grades |
|---|---|---|---|---|
| Febrile neutropenia | | 11 (32) | | 11 (32) |
| Hypophosphataemia | 7 (21) | 3 (9) | | 10 (29) |
| Diarrhea | 9 (26) | | | 9 (26) |
| Fatigue | 6 (18) | 2 (6) | | 8 (24) |
| Oedema peripheral | 8 (24) | | | 8 (24) |
| Rash | 5 (15) | 3 (9) | | 8 (24) |
| Stomatitis | 5 (15) | 3 (9) | | 8 (24) |
| Lung infection | 2 (6) | 5 (15) | | 7 (21) |
| Nausea | 7 (21) | | | 7 (21) |
| Anaemia | | 6 (18) | | 6 (18) |
| Thrombocytopenia | | 2 (6) | 4 (12) | 6 (18) |
| Cough | 5 (15) | | | 5 (15) |
| Epistaxis | 5 (15) | | | 5 (15) |
| Hypertension | 5 (15) | | | 5 (15) |
| Hypocalcaemia | 5 (15) | | | 5 (15) |
| Pleural effusion | 5 (15) | | | 5 (15) |
| Vomiting | 5 (15) | | | 5 (15) |
| Alopecia | 4 (12) | | | 4 (12) |
| Decreased appetite | 3 (9) | 1 (3) | | 4 (12) |
| Dyspnoea | 3 (9) | 1 (3) | | 4 (12) |
| Hypoalbuminaemia | 4 (12) | | | 4 (12) |

Data are number of patients (%)

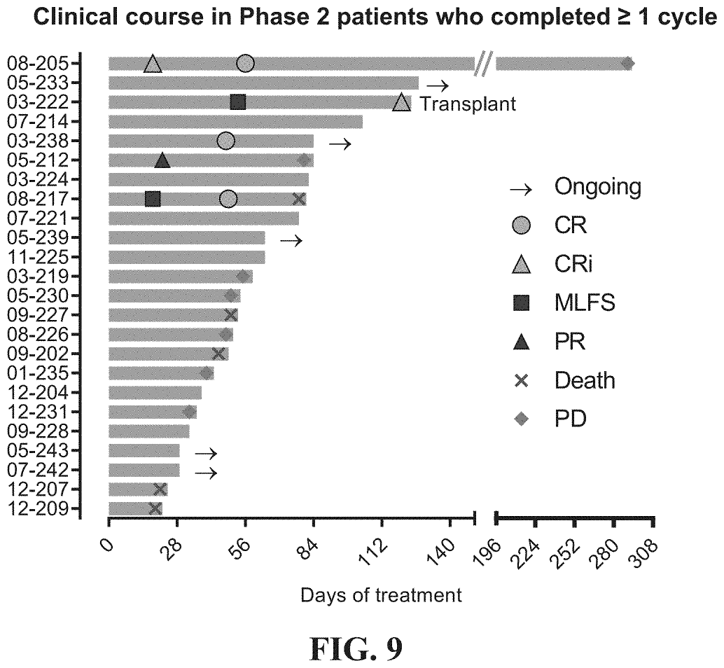

Anti-Leukemic activity: FIG. 9 shows clinical course in phase 2 patients completing ≥1 cycle. Clinical outcome results are shown in Table 21.

TABLE 21
Clinical outcome shown anti-leukemic activity

| | Phase 1b (n-21) | Phase 2 (n = 24) |
|---|---|---|
| CR/CRi; n (%) | 5 (24%) | 4 (17%) |
| CR, CRi, MLFS or PR; n (%) | 7 (33%) | 5 (21%) |
| Number of cycles to achieve | 4 [1-7] | 1.5 [1-4] |
| CR/CRi; median [range] | | |
| Median duration of CR/CRi*; | 17.2 [2.6-20.5] | 1.2 [1-9] |
| months [range] | | |
| Durable response (≥9 months) | 3 | 1 |
| Number of patients with ongoing | 2 | 1 |
| responses, n | | |

*Patients proceeded to transplant after CR/CRi were censored (n = 2, one in Phase 1 and one in Phase 2)

Characteristics of Responders (N=9): (1) Elderly population: median age 73 years [range 51-76]; (2) Prior treatment: (a) 7 (78%) patients had received induction therapy as 1st line therapy, (b) (22%) were treatment naïve for their AML but had received azacitidine for prior MDS; (3) Cytogenetic risk: 6 (67%) patients had adverse cytogenetic risk, 2 (22%) intermediate and 1 favorable (11%); (4) Genomic profiling: (a) 2 patients had no mutation detected at baseline, (b) 5 (55%) patients had a mutation in a splicing factor (SRSF2 or SF3B1). Table 22 shows the number of patients identified with mutation(s) in the corresponding gene (n=number of patients).

TABLE 22
Identification of gene mutations in patients

| Mutation | SRSF2 | SF3B1 | NRAS | ASXL1 | FLT3-ITD |
|---|---|---|---|---|---|
| n | 3 | 2 | 2 | 2 | 2 |
| Mutation | CDKN2A | FLT3-TKD | IDH2 | DNMT3A | |
| n | 1 | 1 | 1 | 1 | |

Decrease in Mutant ctDNA at 1st Cycle is Associated with Clinical Response: For 22 patients, a driver mutation was identified by targeted-NGS at baseline. Mutant allele frequency (MAF) of the selected variant was measured at baseline (C0) and end of cycle 1 (C1) in ctDNA isolated from plasma by NGS or digital droplet PCR. Decrease in mutant ctDNA was associated with clinical response: all the patients with CR/CRi (n=7, 100%) showed a decrease in ctDNA MAF (Log₂(C₁/C₀)<−0.1), while only 2 (13%) of the 15 non-responders showed a similar decrease. The results shown in Table 23 indicate that changes in mutant ctDNA after the first cycle can be used to help guide patient treatment.

TABLE 23
Identified target gene mutations and clinical response

| | Target | ctDNA MAF | Clinical |
|---|---|---|---|
| Patient | Gene | Log₂(C₁/C₀) | Response |
| 07-009 | FLT3 | −0.33 | CR/CRi |
| 07-013 | PHF6 | 0.32 | Non-CR/CRi |
| 07-018 | TP53 | 0 | Non-CR/CRi |
| 01-021 | BCOR | 0.22 | Non-CR/CRi |
| 09-026 | TP53 | 0.35 | Non-CR/CRi |
| 05-030 | NRAS | −0.43 | CR/CRi |
| 07-033 | KRAS | −0.02 | Non-CR/CRi |
| 07-035 | IDH2 | −0.14 | CR/CRi |
| 07-036 | TET2 | 0.29 | Non-CR/CRi |
| 05-043 | NRAS | −0.03 | Non-CR/CRi |
| 08-058 | NRAS | −1.44 | CR/CRi |
| 09-064 | SF3B1 | −1.69 | CR/CRi |
| 09-202 | IDH1 | −0.30 | Non-CR/CRi |
| 12-204 | FLT3 | 0.54 | Non-CR/CRi |
| 08-205 | DNMT3A | −1.21 | CR/CRi |
| 05-212 | NRAS | 0 | Non-CR/CRi |
| 07-214 | KRAS | −0.03 | Non-CR/CRi |
| 08-217 | SRFS2 | −2.5 | CR/CRi |
| 07-221 | SRFS2 | −0.49 | Non-CR/CRi |
| 03-224 | JAK2 | 0.14 | Non-CR/CRi |
| 05-230 | SRFS2 | 0.12 | Non-CR/CRi |
| 05-239 | SRFS2 | −0.13 | Non-CR/CRi |

This example demonstrates that Onvansertib in combination with decitabine is well tolerated. MTD was established at 60 mg/m² with no DLTs occurring through this dose level. Anti-leukemic activity was observed for the combination. CR/CRi rate was 24% in Phase 1b and 17% in Phase 2. Responses were observed in patients with prior induction therapy or MDS disease 55% of responders had a mutation in a splicing factor.

As shown herein, early decreases in mutant ctDNA were associated with clinical response. Therefore, this example further demonstrates that ctDNA can be used an effective biomarker in detecting clinical response in AML treatment and guiding election of treatment options.

### Enumerated Embodiments

1. A method of determining responsiveness of a subject to a leukemia treatment, comprising
analyzing circulating tumor DNA (ctDNA) of a subject with leukemia, wherein the subject is undergoing a treatment and/or has received a treatment for leukemia, thereby determining the responsiveness of the subject to the leukemia treatment.

2. The method of claim 1, wherein determining the responsiveness of the subject comprises determining if the subject is a responder of the treatment, if the subject is or is going to be in CR, if the subject is or is going to be in incomplete hematologic recovery (CRi), if the subject is or is going to be in morphologic leukemia-free state (MLFS), or if the subject is or is going to be in partial remission (PR).

3. The method of any one of claims 1-2, wherein analyzing ctDNA comprises detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, wherein a decrease in the variant allele frequency in at least one of the additional samples relative to the first sample indicates the subject as responsive to the leukemia treatment.

4. The method of any one of claims 1-3, wherein the first time point is prior or immediately prior to the leukemia treatment, and wherein at least one of the one or more additional time points are at the end of or after at least a cycle of the leukemia treatment.

5. The method of claim 4, wherein the cycle of the leukemia treatment is the first cycle of the leukemia treatment.

6. The method of any one of claims 1-3, wherein the first time point is prior or immediately prior to a first cycle of the leukemia treatment, and wherein the one or more additional time points are at the end of or after a second cycle of the leukemia treatment.

7. The method of claim 6, wherein the first cycle of the leukemia treatment is immediately prior to the second cycle of the leukemia treatment.

8. The method of any one of claims 1-7, comprising continuing the leukemia treatment to the subject if the subject is indicated as responsive to the leukemia treatment.

9. The method of any one of claims 1-7, comprising discontinuing the leukemia treatment to the subject and/or starting a different leukemia treatment to the subject if the subject is not indicated as responsive to the leukemia treatment.

10. A method of determining leukemia status of a subject, comprising
analyzing circulating tumor DNA (ctDNA) of a subject, wherein the subject is undergoing a current treatment for leukemia, has received a prior treatment for leukemia, and/or is in remission for leukemia, thereby determining leukemia status of the subject.

11. The method of claim 10, the subject in remission for leukemia is in complete remission (CR), in CR with incomplete hematologic recovery (CRi), in morphologic leukemia-free state (MLFS), or in partial remission (PR).

12. The method of any one of claims 1-11, wherein analyzing the ctDNA comprises detecting variant allele frequency in the ctDNA.

13. The method of any one of claims 10-11, wherein analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, wherein an increase in the variant allele frequency at the additional sample(s) relative to the first sample indicates that the subject is at risk of leukemia relapse or is in leukemia relapse.

14. The method of claim 13, wherein the first time point is prior or immediately prior to the leukemia treatment, and wherein the one or more additional time points are at the end of or after at least a cycle of the leukemia treatment, optionally wherein the cycle of the leukemia treatment is the first cycle of the leukemia treatment.

15. The method of claim 13, wherein the first time point is prior or immediately prior to a first cycle of the leukemia treatment, and wherein the one or more additional time points are at the end of or after a second cycle of the leukemia treatment, optionally wherein the first cycle of the leukemia treatment is immediately prior to the second cycle of the leukemia treatment.

16. The method of any one of claims 13-15, comprising starting an additional leukemia treatment to the subject if the subject is indicated as in leukemia relapse.

17. The method of claim 16, wherein the additional leukemia treatment is different from the current or prior leukemia treatment.

18. The method of any one of claims 3-9 and 13-17, wherein the variant allele frequency in ctDNA is determined by total mutation count in the ctDNA in each of the first sample and one or more additional samples.

19. The method of any one of claims 3-9 and 13-17, wherein the variant allele frequency in ctDNA is determined by the mean variant allele frequency in each of the first sample and one or more additional samples.

20. The method of any one of claims 3-9 and 13-19, wherein the variant allele frequency is mutant allelic frequency (MAF) for a driver mutation of leukemia.

21. The method of any one of claims 3-9 and 13-19, wherein the variant allele frequency is mutant allelic frequency (MAF) for one or more driver mutations of leukemia.

22. The method of any one of claims 20-21, wherein Log₂(C₁/C₀)<a MAF threshold indicates a decrease in ctDNA MAF wherein C₀ is ctDNA MAF in the first sample and C₁ is ctDNA MAF in one of the additional samples.

23. The method of claim 22, wherein the MAF threshold is −0.06.

24. The method of any one of claims 1-23, wherein the first sample comprises ctDNA from the subject before treatment, and the one of additional samples comprises ctDNA from the subject after treatment.

25. The method of any one of claims 20-24, wherein the driver mutation is a mutation in one of the 75 genes set forth in Table 3, wherein at least one of the one or more the driver mutations is a mutation in in the 75 genes set forth in Table 3, and/or wherein one or more the driver mutations are mutations in the 75 genes set forth in Table 3.

26. The method of any one of claims 20-24, wherein the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1.

27. The method of any one of claims 20-24, wherein the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

28. The method of any one of claims 20-24, wherein the driver mutation or at least one of the one or more driver mutations is one or more of the mutations set forth in Table 12 as HGV Sc.

29. The method any one of claims 3-9 and 13-28, further comprising determining variant allele frequency in one or more of the ctDNA, PBMCs and BMMCs of the subject.

30. The method of any one of claims 1-29, wherein the ctDNA is analyzed using polymerase chain reaction (PCR) or next generation sequencing (NGS).

31. The method of any one of claims 1-29, wherein the ctDNA is analyzed using droplet digital PCR (ddPCR).

32. The method of any one of claims 1-31, wherein the leukemia is advanced, metastatic, refractory, and/or relapsed

33. The method of any one of claims 1-31, wherein the leukemia is acute myeloid leukemia (AML), myelodysplastic syndrome (MDS) or chronic melomonocytic leukemia (CMML).

34. The method of any one of claims 1-31, wherein the leukemia is relapsed or refractory acute myeloid leukemia.

35. The method of any one of claim 1-34, wherein the sample is derived from whole blood of the subject, plasma of the subject, serum of the subject, or a combination thereof.

36. The method of any one of claim 1-34, wherein the ctDNA is from whole blood of the subject, plasma of the subject, serum of the subject, or a combination thereof.

37. The method of any one of claims 1-36, wherein the treatment comprises standard of care therapies for leukemia.

38. The method of any one of claims 1-36, wherein the treatment comprises administration of a Polo-like kinase 1 (PLK1) inhibitor.

39. The method of claim 38, wherein the administration of PLK1 inhibitor is oral administration.

40. The method of claim 38 or 39, wherein the PLK1 inhibitor is onvansertib.

41. The method of claim 40, wherein the treatment comprises administration of onvansertib for five days in a cycle of 21 to 28 days.

42. The method of claim 41, wherein the treatment comprises administration of onvansertib at 12 mg/m²-90 mg/m².

43. The method of any one of claims 40-42, wherein a maximum concentration (Cₘₐₓ) of onvansertib in a blood of the subject is from about 100 nmol/L to about 1500 nmol/L.

44. The method of any one of claims 40-43, wherein an area under curve (AUC) of a plot of a concentration of onvanserib in a blood of the subject over time is from about 1000 nmol/L·hour to about 400000 nmol/L·hour.

45. The method of any one of claims 40-44, wherein a time (Tₘₐₓ) to reach a maximum concentration of onvansertib in a blood of the subject is from about 1 hour to about 5 hours.

46. The method of any one of claims 40-45, wherein an elimination half-life (T_{1/2}) of onvansertib in a blood of the subject is from about 10 hours to about 60 hours

47. The method of any one of claims 38-46, wherein the treatment comprises at least one additional administration of cancer therapeutics or cancer therapy.

48. The method of claim 47, wherein the PLK inhibitor and the cancer therapeutics or cancer therapy are co-administered simultaneously or sequentially.

49. The method of any one of claims 47-48, wherein the additional cancer therapeutics is cytarabine, low-dose cytarabine (LDAC) and/or decitabine.

50. The method of claim 49, wherein the treatment comprises administration of LDAC at 20 mg/m² subcutaneous (SC) once a day (qd) for ten days in a cycle, and/or wherein the treatment comprises administration of decitabine at 20 mg/m² intravenous (IV) qd for five days in a cycle.

51. The method of any one of claims 1-50, further comprising analyzing ctDNA of the subject before the treatment.

52. The method of any one of claim 1-51, wherein the treatment comprises one or more cycles, and the ctDNA is analyzed before, during and after each cycle of the treatment.

53. The method of claim 52, wherein each cycle of treatment is at least 21 days. 54. The method of claim 52, wherein each cycle of treatment is from about 21 days to about 28 days.

55. The method of any one of claims 1-54, the subject is human.

56. A method of improving treatment outcome for leukemia, comprising
detecting variant allele frequency in circulating tumor DNA (ctDNA) obtained from a subject at a first time point in a first sample before the subject undergoes a leukemia treatment;
detecting variant allele frequency in ctDNA obtained from the subject at one or more additional time points in one or more additional samples after the subject undergoes the leukemia treatment;
determining the difference of the variant allele frequency in ctDNA between the first and at least one of the one or more additional samples, wherein a decrease in the variant allele frequency in at least one of the additional samples relative to the first sample indicates the subject as responsive to the leukemia treatment; and
continuing the leukemia treatment to the subject if the subject is indicated as responsive to the leukemia treatment, or discontinuing the leukemia treatment to the subject and/or starting a different leukemia treatment to the subject if the subject is not indicated as responsive to the leukemia treatment.

57. A method of treating leukemia, comprising:
administering a leukemia treatment to a subject in need thereof;
determining a decrease, relative to a variant allele frequency in a first sample of the subject obtained at a first time point before the subject receives the leukemia treatment, in a variant allele frequency in a second sample of the subject obtained at a second time point after the subject receives the leukemia treatment; and
continuing with the leukemia treatment.

58. The method of any one of claims 56-57, wherein the subject is a subject newly diagnosed with leukemia.

59. The method of any one of claims 56-58, wherein the subject has not received any prior cancer treatment before the leukemia treatment.

60. The method of any one of claims 56-57, the subject has received prior cancer treatment and was in remission for leukemia.

61. The method of claim 60, wherein the subject was in complete remission (CR), in CR with incomplete hematologic recovery (CRi), in morphologic leukemia-free state (MLFS), or in partial remission (PR) after receiving the prior cancer treatment.

62. The method of any one of claims 56-61, wherein the first time point is prior or immediately prior to the leukemia treatment, and wherein at least one of the one or more additional time points are at the end of or after at least a cycle of the leukemia treatment.

63. The method of claim 62, wherein the cycle of the leukemia treatment is the first cycle of the leukemia treatment.

64. The method of any one of claims 56-63, wherein the first time point is prior or immediately prior to a first cycle of the leukemia treatment, and wherein the one or more additional time points are at the end of or after a second cycle of the leukemia treatment.

65. The method of claim 64, wherein the first cycle of the leukemia treatment is immediately prior to the second cycle of the leukemia treatment.

66. The method of any one of claims 56-65, wherein the variant allele frequency in ctDNA is determined by total mutation count in the ctDNA in each of the first sample and one or more additional samples.

67. The method of any one of claims 56-65, wherein the variant allele frequency in ctDNA is determined by the mean variant allele frequency in each of the first sample and one or more additional samples.

68. The method of any one of claims 56-67, wherein the variant allele frequency is mutant allelic frequency (MAF) for a driver mutation of leukemia.

69. The method of any one of claims 56-67, wherein the variant allele frequency is mutant allelic frequency (MAF) for one or more driver mutations of leukemia.

70. The method of any one of claims 68-69, wherein Log₂(C₁/C₀)<a MAF threshold indicates a decrease in ctDNA MAF wherein C₀ is ctDNA MAF in the first sample and C₁ is ctDNA MAF in one of the additional samples.

71. The method of claim 70, wherein the MAF threshold is −0.06.

72. The method of any one of claims 68-71, wherein the driver mutation is a mutation in one of the 75 genes set forth in Table 3, wherein at least one of the one or more the driver mutations is a mutation in in the 75 genes set forth in Table 3, and/or wherein one or more the driver mutations are mutations in the 75 genes set forth in Table 3.

73. The method of any one of claims 68-71, wherein the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1.

74. The method of any one of claims 68-71, wherein the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

75. The method of any one of claims 68-71, wherein the driver mutation or at least one of the one or more driver mutations is one or more of the mutations set forth in Table 12 as HGV Sc.

76. The method any one of claims 56-75, further comprising determining variant allele frequency in one or more of the ctDNA, PBMCs and BMMCs of the subject.

77. The method of any one of claims 56-76, wherein the variant allele frequency in ctDNA is detected using polymerase chain reaction (PCR) or next generation sequencing (NGS).

78. The method of any one of claims 56-76, wherein the variant allele frequency in ctDNA is detected using droplet digital PCR (ddPCR).

79. The method of any one of claims 56-78, wherein the leukemia is advanced, metastatic, refractory, and/or relapsed.

80. The method of any one of claims 56-79, wherein the leukemia is acute myeloid leukemia (AML), myelodysplastic syndrome (MDS) or chronic melomonocytic leukemia (CMML).

81. The method of any one of claims 56-79, wherein the leukemia is relapsed or refractory acute myeloid leukemia.

82. The method of any one of claim 56-81, wherein at least one of the first sample, the one or more additional samples, and the second sample is derived from whole blood of the subject, plasma of the subject, serum of the subject, or a combination thereof.

83. The method of any one of claim 56-82, wherein the ctDNA is from whole blood of the subject, plasma of the subject, serum of the subject, or a combination thereof.

84. The method of any one of claims 56-83, wherein the leukemia treatment comprises standard of care therapies for leukemia.

85. The method of any one of claims 56-84, wherein the leukemia treatment comprises administration of a Polo-like kinase 1 (PLK1) inhibitor.

86. The method of claim 85, wherein the administration of PLK1 inhibitor is oral administration.

87. The method of claim 85 or 86, wherein the PLK1 inhibitor is onvansertib.

88. The method of claim 87, wherein the treatment comprises administration of onvansertib for five days in a cycle of 21 to 28 days.

89. The method of claim 87 or 88, wherein the treatment comprises administration of onvansertib at 12 mg/m²-90 mg/m².

90. The method of any one of claims 87-89, wherein a maximum concentration (Cₘₐₓ) of onvansertib in a blood of the subject is from about 100 nmol/L to about 1500 nmol/L.

91. The method of any one of claims 87-90, wherein an area under curve (AUC) of a plot of a concentration of onvanserib in a blood of the subject over time is from about 1000 nmol/L·hour to about 400000 nmol/L·hour.

92. The method of any one of claims 87-91, wherein a time (Tₘₐₓ) to reach a maximum concentration of onvansertib in a blood of the subject is from about 1 hour to about 5 hours.

93. The method of any one of claims 87-92, wherein an elimination half-life (T_{1/2}) of onvansertib in a blood of the subject is from about 10 hours to about 60 hours.

94. The method of any one of claims 85-93, wherein the leukemia treatment comprises at least one additional administration of cancer therapeutics or cancer therapy.

95. The method of claim 94, wherein the PLK inhibitor and the cancer therapeutics or cancer therapy are co-administered simultaneously or sequentially.

96. The method of any one of claims 94-95, wherein the additional cancer therapeutics is cytarabine, low-dose cytarabine (LDAC) and/or decitabine.

97. The method of claim 96, wherein the leukemia treatment comprises administration of LDAC at 20 mg/m² subcutaneous (SC) once a day (qd) for ten days in a cycle, and/or wherein the treatment comprises administration of decitabine at 20 mg/m² intravenous (IV) qd for five days in a cycle.

98. The method of any one of claims 56-97, wherein the leukemia treatment comprises one or more cycles, and variant allele frequency in ctDNA is detected before, during and after each cycle of the leukemia treatment.

99. The method of claim 98, wherein each cycle of treatment is at least 21 days.

100. The method of claim 98, wherein each cycle of treatment is from about 21 days to about 28 days.

101. The method of any one of claims 56-100, wherein the subject is human.

102. Use of a PLK1 inhibitor as a treatment of a subject with leukemia, wherein the responsiveness of the subject to the treatment is determined using a method of any one of claims 1-9 and 18-55, wherein the leukemia status of the subject is determined using a method of any one of claims 10-55, wherein the treatment outcome is improved using a method of any one of claims 56-101, and/or wherein the subject is treated using a method of any one of claims 57-101.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of determining responsiveness of a subject to a leukemia treatment, comprising
analyzing circulating tumor DNA (ctDNA) of a subject with leukemia, wherein the subject is undergoing a treatment and/or has received a treatment for leukemia, thereby determining the responsiveness of the subject to the leukemia treatment.

2. The method of claim 1, wherein determining the responsiveness of the subject comprises determining if the subject is a responder of the treatment, if the subject is or is going to be in CR, if the subject is or is going to be in incomplete hematologic recovery (CRi), if the subject is or is going to be in morphologic leukemia-free state (MLFS), or if the subject is or is going to be in partial remission (PR).

3. The method of claim 1, wherein analyzing ctDNA comprises detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, wherein a decrease in the variant allele frequency in at least one of the additional samples relative to the first sample indicates the subject as responsive to the leukemia treatment.

4. The method of claim 1, wherein the first time point is prior or immediately prior to the leukemia treatment, and wherein at least one of the one or more additional time points are at the end of or after at least a cycle of the leukemia treatment.

5. The method of claim 4, wherein the cycle of the leukemia treatment is the first cycle of the leukemia treatment.

6. The method of claim 1, wherein the first time point is prior or immediately prior to a first cycle of the leukemia treatment, and wherein the one or more additional time points are at the end of or after a second cycle of the leukemia treatment.

7. The method of claim 6, wherein the first cycle of the leukemia treatment is immediately prior to the second cycle of the leukemia treatment.

8. The method of claim 1, comprising continuing the leukemia treatment to the subject if the subject is indicated as responsive to the leukemia treatment.

9. The method of claim 1, comprising discontinuing the leukemia treatment to the subject and/or starting a different leukemia treatment to the subject if the subject is not indicated as responsive to the leukemia treatment.

10. A method of determining leukemia status of a subject, comprising
analyzing circulating tumor DNA (ctDNA) of a subject, wherein the subject is undergoing a current treatment for leukemia, has received a prior treatment for leukemia, and/or is in remission for leukemia, thereby determining leukemia status of the subject.

11. The method of claim 10, the subject in remission for leukemia is in complete remission (CR), in CR with incomplete hematologic recovery (CRi), in morphologic leukemia-free state (MLFS), or in partial remission (PR).

12. The method of claim 11, wherein analyzing the ctDNA comprises detecting variant allele frequency in the ctDNA.

13. The method of claim 10, wherein analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, wherein an increase in the variant allele frequency at the additional sample(s) relative to the first sample indicates that the subject is at risk of leukemia relapse or is in leukemia relapse.

14. The method of claim 13, wherein the first time point is prior or immediately prior to the leukemia treatment, and wherein the one or more additional time points are at the end of or after at least a cycle of the leukemia treatment, optionally wherein the cycle of the leukemia treatment is the first cycle of the leukemia treatment.

15. The method of claim 13, wherein the first time point is prior or immediately prior to a first cycle of the leukemia treatment, and wherein the one or more additional time points are at the end of or after a second cycle of the leukemia treatment, optionally wherein the first cycle of the leukemia treatment is immediately prior to the second cycle of the leukemia treatment.

16. The method of claim 13, comprising starting an additional leukemia treatment to the subject if the subject is indicated as in leukemia relapse.

17. The method of claim 16, wherein the additional leukemia treatment is different from the current or prior leukemia treatment.

18. The method of claim 3, wherein the variant allele frequency in ctDNA is determined by total mutation count in the ctDNA in each of the first sample and one or more additional samples.

19. The method of claim 3, wherein the variant allele frequency in ctDNA is determined by the mean variant allele frequency in each of the first sample and one or more additional samples.

20. The method of claim 3, wherein the variant allele frequency is mutant allelic frequency (MAF) for a driver mutation of leukemia.

21. The method of claim 3, wherein the variant allele frequency is mutant allelic frequency (MAF) for one or more driver mutations of leukemia.

22. The method of claim 20, wherein Log₂(C₁/C₀)<a MAF threshold indicates a decrease in ctDNA MAF wherein C₀ is ctDNA MAF in the first sample and C₁ is ctDNA MAF in one of the additional samples.

23. The method of claim 22, wherein the MAF threshold is −0.06.

24. The method of claim 1, wherein the first sample comprises ctDNA from the subject before treatment, and the one of additional samples comprises ctDNA from the subject after treatment.

25. The method of claim 20, wherein the driver mutation is a mutation in one of the 75 genes set forth in Table 3, wherein at least one of the one or more the driver mutations is a mutation in in the 75 genes set forth in Table 3, and/or wherein one or more the driver mutations are mutations in the 75 genes set forth in Table 3.

26. The method of claim 20, wherein the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1.

27. The method of claim 20, wherein the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

28. The method of claim 20, wherein the driver mutation or at least one of the one or more driver mutations is one or more of the mutations set forth in Table 12 as HGVSc.

29. The method claim 3, further comprising determining variant allele frequency in one or more of the ctDNA, PBMCs and BMMCs of the subject.

30. The method of claim 1, wherein the ctDNA is analyzed using polymerase chain reaction (PCR) or next generation sequencing (NGS).

31. The method of claim 1, wherein the ctDNA is analyzed using droplet digital PCR (ddPCR).

32. The method of claim 1, wherein the leukemia is advanced, metastatic, refractory, and/or relapsed

33. The method of claim 1, wherein the leukemia is acute myeloid leukemia (AML), myelodysplastic syndrome (MDS) or chronic melomonocytic leukemia (CMML).

34. The method of claim 1, wherein the leukemia is relapsed or refractory acute myeloid leukemia.

35. The method of claim 1, wherein the sample is derived from whole blood of the subject, plasma of the subject, serum of the subject, or a combination thereof.

36. The method of claim 1, wherein the ctDNA is from whole blood of the subject, plasma of the subject, serum of the subject, or a combination thereof.

37. The method of claim 1, wherein the treatment comprises standard of care therapies for leukemia.

38. The method of claim 1, wherein the treatment comprises administration of a Polo-like kinase 1 (PLK1) inhibitor.

39. The method of claim 38, wherein the administration of PLK1 inhibitor is oral administration.

40. The method of claim 38, wherein the PLK1 inhibitor is onvansertib.

41. The method of claim 40, wherein the treatment comprises administration of onvansertib for five days in a cycle of 21 to 28 days.

42. The method of claim 41, wherein the treatment comprises administration of onvansertib at 12 mg/m²-90 mg/m².

43. The method of claim 40, wherein a maximum concentration (Cₘₐₓ) of onvansertib in a blood of the subject is from about 100 nmol/L to about 1500 nmol/L.

44. The method of claim 40, wherein an area under curve (AUC) of a plot of a concentration of onvanserib in a blood of the subject over time is from about 1000 nmol/L·hour to about 400000 nmol/L·hour.

45. The method of claim 40, wherein a time (Tₘₐₓ) to reach a maximum concentration of onvansertib in a blood of the subject is from about 1 hour to about 5 hours.

46. The method of claim 40, wherein an elimination half-life (T_{1/2}) of onvansertib in a blood of the subject is from about 10 hours to about 60 hours

47. The method of claim 38, wherein the treatment comprises at least one additional administration of cancer therapeutics or cancer therapy.

48. The method of claim 47, wherein the PLK inhibitor and the cancer therapeutics or cancer therapy are co-administered simultaneously or sequentially.

49. The method of claim 47, wherein the additional cancer therapeutics is cytarabine, low-dose cytarabine (LDAC) and/or decitabine.

50. The method of claim 49, wherein the treatment comprises administration of LDAC at 20 mg/m² subcutaneous (SC) once a day (qd) for ten days in a cycle, and/or wherein the treatment comprises administration of decitabine at 20 mg/m² intravenous (IV) qd for five days in a cycle.

51. The method of claim 1, further comprising analyzing ctDNA of the subject before the treatment.

52. The method of claim 1, wherein the treatment comprises one or more cycles, and the ctDNA is analyzed before, during and after each cycle of the treatment.

53. The method of claim 52, wherein each cycle of treatment is at least 21 days.

54. The method of claim 52, wherein each cycle of treatment is from about 21 days to about 28 days.

55. The method of claim 1, the subject is human.

56. A method of improving treatment outcome for leukemia, comprising
detecting variant allele frequency in circulating tumor DNA (ctDNA) obtained from a subject at a first time point in a first sample before the subject undergoes a leukemia treatment;
detecting variant allele frequency in ctDNA obtained from the subject at one or more additional time points in one or more additional samples after the subject undergoes the leukemia treatment;
determining the difference of the variant allele frequency in ctDNA between the first and at least one of the one or more additional samples, wherein a decrease in the variant allele frequency in at least one of the additional samples relative to the first sample indicates the subject as responsive to the leukemia treatment; and
continuing the leukemia treatment to the subject if the subject is indicated as responsive to the leukemia treatment, or discontinuing the leukemia treatment to the subject and/or starting a different leukemia treatment to the subject if the subject is not indicated as responsive to the leukemia treatment.

57. A method of treating leukemia, comprising:
administering a leukemia treatment to a subject in need thereof;
determining a decrease, relative to a variant allele frequency in a first sample of the subject obtained at a first time point before the subject receives the leukemia treatment, in a variant allele frequency in a second sample of the subject obtained at a second time point after the subject receives the leukemia treatment; and
continuing with the leukemia treatment.

58. The method of claim 56, wherein the subject is a subject newly diagnosed with leukemia.

59. The method of claim 56, wherein the subject has not received any prior cancer treatment before the leukemia treatment.

60. The method of claim 56, the subject has received prior cancer treatment and was in remission for leukemia.

61. The method of claim 60, wherein the subject was in complete remission (CR), in CR with incomplete hematologic recovery (CRi), in morphologic leukemia-free state (MLFS), or in partial remission (PR) after receiving the prior cancer treatment.

62. The method of claim 56, wherein the first time point is prior or immediately prior to the leukemia treatment, and wherein at least one of the one or more additional time points are at the end of or after at least a cycle of the leukemia treatment.

63. The method of claim 62, wherein the cycle of the leukemia treatment is the first cycle of the leukemia treatment.

64. The method of claim 56, wherein the first time point is prior or immediately prior to a first cycle of the leukemia treatment, and wherein the one or more additional time points are at the end of or after a second cycle of the leukemia treatment.

65. The method of claim 64, wherein the first cycle of the leukemia treatment is immediately prior to the second cycle of the leukemia treatment.

66. The method of claim 56, wherein the variant allele frequency in ctDNA is determined by total mutation count in the ctDNA in each of the first sample and one or more additional samples.

67. The method of claim 56, wherein the variant allele frequency in ctDNA is determined by the mean variant allele frequency in each of the first sample and one or more additional samples.

68. The method of claim 56, wherein the variant allele frequency is mutant allelic frequency (MAF) for a driver mutation of leukemia.

69. The method of claim 56, wherein the variant allele frequency is mutant allelic frequency (MAF) for one or more driver mutations of leukemia.

70. The method of claim 68, wherein Log₂(C₁/C₀)<a MAF threshold indicates a decrease in ctDNA MAF wherein C₀ is ctDNA MAF in the first sample and C₁ is ctDNA MAF in one of the additional samples.

71. The method of claim 70, wherein the MAF threshold is −0.06.

72. The method of claim 68, wherein the driver mutation is a mutation in one of the 75 genes set forth in Table 3, wherein at least one of the one or more the driver mutations is a mutation in in the 75 genes set forth in Table 3, and/or wherein one or more the driver mutations are mutations in the 75 genes set forth in Table 3.

73. The method of claim 68, wherein the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1.

74. The method of claim 68, wherein the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

75. The method of claim 68, wherein the driver mutation or at least one of the one or more driver mutations is one or more of the mutations set forth in Table 12 as HGVSc.

76. The method of claim 56, further comprising determining variant allele frequency in one or more of the ctDNA, PBMCs and BMMCs of the subject.

77. The method of claim 56, wherein the variant allele frequency in ctDNA is detected using polymerase chain reaction (PCR) or next generation sequencing (NGS).

78. The method of claim 56, wherein the variant allele frequency in ctDNA is detected using droplet digital PCR (ddPCR).

79. The method of claim 56, wherein the leukemia is advanced, metastatic, refractory, and/or relapsed.

80. The method of claim 56, wherein the leukemia is acute myeloid leukemia (AML), myelodysplastic syndrome (MDS) or chronic melomonocytic leukemia (CMML).

81. The method of claim 56, wherein the leukemia is relapsed or refractory acute myeloid leukemia.

82. The method of claim 56, wherein at least one of the first sample, the one or more additional samples, and the second sample is derived from whole blood of the subject, plasma of the subject, serum of the subject, or a combination thereof.

83. The method of claim 56, wherein the ctDNA is from whole blood of the subject, plasma of the subject, serum of the subject, or a combination thereof.

84. The method of claim 56, wherein the leukemia treatment comprises standard of care therapies for leukemia.

85. The method of claim 56, wherein the leukemia treatment comprises administration of a Polo-like kinase 1 (PLK1) inhibitor.

86. The method of claim 85, wherein the administration of PLK1 inhibitor is oral administration.

87. The method of claim 85, wherein the PLK1 inhibitor is onvansertib.

88. The method of claim 87, wherein the treatment comprises administration of onvansertib for five days in a cycle of 21 to 28 days.

89. The method of claim 87, wherein the treatment comprises administration of onvansertib at 12 mg/m²-90 mg/m².

90. The method of claim 87, wherein a maximum concentration (Cₘₐₓ) of onvansertib in a blood of the subject is from about 100 nmol/L to about 1500 nmol/L.

91. The method of claim 87, wherein an area under curve (AUC) of a plot of a concentration of onvanserib in a blood of the subject over time is from about 1000 nmol/L·hour to about 400000 nmol/L·hour.

92. The method of claim 87, wherein a time (Tₘₐₓ) to reach a maximum concentration of onvansertib in a blood of the subject is from about 1 hour to about 5 hours.

93. The method of claim 87, wherein an elimination half-life (T_{1/2}) of onvansertib in a blood of the subject is from about 10 hours to about 60 hours.

94. The method of claim 85, wherein the leukemia treatment comprises at least one additional administration of cancer therapeutics or cancer therapy.

95. The method of claim 94, wherein the PLK inhibitor and the cancer therapeutics or cancer therapy are co-administered simultaneously or sequentially.

96. The method of claim 94, wherein the additional cancer therapeutics is cytarabine, low-dose cytarabine (LDAC) and/or decitabine.

97. The method of claim 96, wherein the leukemia treatment comprises administration of LDAC at 20 mg/m² subcutaneous (SC) once a day (qd) for ten days in a cycle, and/or wherein the treatment comprises administration of decitabine at 20 mg/m² intravenous (IV) qd for five days in a cycle.

98. The method of claim 56, wherein the leukemia treatment comprises one or more cycles, and variant allele frequency in ctDNA is detected before, during and after each cycle of the leukemia treatment.

99. The method of claim 98, wherein each cycle of treatment is at least 21 days.

100. The method of claim 98, wherein each cycle of treatment is from about 21 days to about 28 days.

101. The method of claim 56, wherein the subject is human.
